(19) United States
(12) Patent Application Publication
(10) Pub. No.: US 2025/0034265 A1
(43) Pub. Date: Jan. 30, 2025

(54) TREATMENT OF A DEMYELINATING DISEASE OF THE CENTRAL NERVOUS SYSTEM (CNS) WITH SATRALIZUMAB

(71) Applicants: Chugai Seiyaku Kabushiki Kaisha, Tokyo (JP); F. Hoffmann-La Roche AG, Basel (CH)

(72) Inventors: Takatoshi Ozawa, Tokyo (JP); Mai Yamashiro, Tokyo (JP); Hajime Ito, Tokyo (JP); Shunsuke Yoshida, Tokyo (JP); Jillian Smith, Welwyn Garden City (GB); Ivana Vodopivec, Basel (CH); Sian Lennon-Chrimes, Welwyn Garden City (GB); Gaelle Klingelschmitt, Basel (CH); Hans-Christian Von Buedingen, Basel (CH); Hanna Silber Baumann, Basel (CH)

(73) Assignees: Chugai Seiyaku Kabushiki Kaisha, Tokyo (JP); F. Hoffmann-La Roche AG, Basel (CH)

(21) Appl. No.: 18/712,917

(22) PCT Filed: Nov. 25, 2022
(86) PCT No.: PCT/JP2022/043437

§ 371 Date: May 23, 2024

(30) Foreign Application Priority Data
Nov. 26, 2021 (WO) PCT/JP2021/043459
Oct. 25, 2022 (WO) PCT/JP2022/039605

(51) Int. Cl. C07K 16/28; A61K 39/00; A61P 37/06

(52) U.S. Cl. CPC C07K 16/2866; A61P 37/06; A61K 2039/505; C07K 2317/24; C07K 2317/76

(57) ABSTRACT

The invention provides a means for a treatment for a demyelinating disease of the central nervous system (CNS) characterized by the presence of an anti-myelin oligodendrocyte glycoprotein (MOG) antibody, and also for reducing the risk of relapse in the demyelinating disease, comprising an anti-IL-6 receptor antibody or antigen binding fragment thereof.

### CROSS REFERENCE TO RELATED APPLICATIONS

This application is the National Stage of International Application No. PCT/JP2022/043437, filed on Nov. 25, 2022, which claims the benefit of International Application Nos. PCT/JP2021/043459, filed on Nov. 26, 2021, and PCT/JP2022/039605, filed on Oct. 25, 2022. The contents of PCT/JP2022/043437 are incorporated herein by reference in their entirety.

This application contains a sequence listing that has been submitted electronically as an XML file named “C1-X2117PPPsq.xml.” The XML file was created on Nov. 4, 2022, and is 50,662 bytes in size. The material in the XML file is hereby incorporated by reference in its entirety.

### TECHNICAL FIELD

The present invention relates to a medicament or a pharmaceutical composition for treatment, or for reducing the risk of relapse, of a demyelinating disease of the central nervous system (CNS) that is characterized by the presence of an anti-myelin oligodendrocyte glycoprotein (MOG) antibody, the composition comprising an anti-IL-6 receptor antibody or antigen binding fragment thereof. The present invention also relates to a method of treatment, or of reducing the risk of relapse, of said demyelinating disease by administering an anti-IL-6 receptor antibody or antigen binding fragment thereof to a subject in need thereof.

### BACKGROUND ART

Myelin oligodendrocyte glycoprotein antibody-associated disease (MOGAD) is a rare autoimmune demyelinating disease of the CNS characterized by the presence of anti-myelin oligodendrocyte glycoprotein antibodies (MOG-IgG) in adults and children. MOG is a transmembrane protein expressed on oligodendrocytes and the outer layers of myelin sheath [NPL 19]. The disease is characterized by attacks of optic neuritis, transverse myelitis, brain or brainstem inflammation, or combinations thereof (NPL 1). A combination of a compatible clinical and radiologic phenotype and seropositivity for MOG-IgG is required to establish the diagnosis. In about 80% of adult patients, the disease is chronic, characterized by a relapsing course (NPL 2, NPL 3, and NPL 4). The proportion of adolescents with relapsing disease course is believed to be similar to adults (NPL 5 and NPL 6). MOGAD-associated disability is attack/relapse-driven, hence the importance of relapse prevention. There are no approved therapies for MOGAD, and consensus-based treatment guidelines are missing. MOGAD is worsened by several multiple sclerosis (MS) disease-modifying treatments, including interferon-beta (IFN-beta), glatiramer acetate, teriflunomide, dimethyl fumarate, cladribine, fingolimod, natalizumab, and alemtuzumab (NPL 7, NPL 8, NPL 9, and NPL 6). The current MOGAD treatment paradigm includes the use of corticosteroids with or without intravenous immunoglobulins (IVIg) or plasma exchange (PLEX) for acute treatment of attacks, and empirically selected conventional steroid-sparing immunosuppressant treatments (ISTs) and rituximab (RTX) for relapse prevention (NPL 10, NPL 9, NPL 11, NPL 12, and NPL 13). Most recent literature indicates that these medications, which are associated with numerous short and long-term adverse effects, are often only partially effective (NPL 9, NPL 14, NPL 15, and NPL 6). There continues to be a need for safe, provenly effective and convenient chronic treatments for MOGAD.

There is no approved treatment for MOGAD or for prevention of MOGAD relapses. ISTs used empirically off-label are often only partially effective, and many are associated with numerous short- and long-term adverse effects. Recently, elevated interleukin (IL)-6 levels in the cerebrospinal fluid (CSF) and serum have been reported in patients with MOGAD (NPL 16). There are some reports relating to the off-label use of tocilizumab, an anti-IL-6 receptor antibody in patients with MOGAD. However, the exact role of IL-6 in MOGAD is unclear. (NPL 17, NPL 18, and NPL 19).

Humanized antibodies like tocilizumab are first-generation antibody drugs. By improving first-generation antibody drugs, second-generation antibody drugs with improved efficacy, convenience, and cost are being developed (PTL 2 and PTL 3). Among the second-generation antibody drugs is satralizumab (SA237), which is a novel anti-IL-6 receptor antibody to which improvement technologies such as enhancement of antigen-binding ability, pharmacokinetics, and stability, and reduction of immunogenicity risk, have been applied (PTL 3 and PTL 4).

Satralizumab is a humanized anti-IL-6 receptor monoclonal antibody with pH-dependent antigen binding. It specifically targets the human IL-6 receptor (IL-6R) and suppresses IL-6 signaling by inhibiting the binding of IL-6 to membrane-bound IL-6R and soluble IL-6R. Satralizumab was constructed by modifying the amino acid sequence of tocilizumab to prolong its plasma half-life. Satralizumab also shows a decreased antibody molecule isoelectric point and stronger binding to FcRn compared to tocilizumab. Moreover, its Fc region has been modified to minimize the antibody-dependent cellular cytotoxicity and complement-dependent cytotoxic effector activity compared to tocilizumab.

Prior-art literature information related to the invention of the present application is shown below.

### CITATION LIST

### Patent Literature

[PTL 1] US2012/0039840
[PTL 2] WO2009/041621
[PTL 3] WO2010/035769
[PTL 4] WO2016/136933

### Non-Patent Literature

[NPL 1] Lopez-Chiriboga A S, Majed M, Fryer J, et al. Association of MOG-IgG Serostatus With Relapse After Acute Disseminated Encephalomyelitis and Proposed Diagnostic Criteria for MOG-IgG-Associated Disorders. JAMA Neurol. 2018 Nov. 1; 75(11):1355-1363.
[NPL 2] Jarius S, Ruprecht K, Kleiter I, et al. MOG-IgG in NMO and related disorders: a multicenter study of 50 patients. Part 2: Epidemiology, clinical presentation, radiological and laboratory features, treatment responses, and long-term outcome. J Neuroinflammation. 2016; 13(1):280.
[NPL 3] Hyun J W, Woodhall M R, Kim S H, et al. Longitudinal analysis of myelin oligodendrocyte glycoprotein antibodies in CNS inflammatory diseases. J Neurol Neurosurg Psychiatry. 2017; 88(10):811-817.
[NPL 4] Salama S, Pardo S, Levy M. Clinical characteristics of myelin oligodendrocyte glycoprotein antibody neuromyelitis optica spectrum disorder. Mult Scler Relat Disord. 2019; 30:231-235.
[NPL 5] Bruijstens A L, Breu M, Wendel E-M, et al. E.U. paediatric MOG consortium consensus: Part 4—Outcome of paediatric myelin oligodendrocyte glycoprotein antibody-associated disorders. Eur J Paediatr Neurol 2020b; 29:32-40.
[NPL 6] Cobo-Calvo A, Ruiz A, Rollot F, et al. Clinical Features and Risk of Relapse in Children and Adults with Myelin Oligodendrocyte Glycoprotein Antibody-Associated Disease. Ann Neurol. 2021; 89(1):30-41.
[NPL 7] Wildemann B, Janus S, Schwarz A, et al. Failure of alemtuzumab therapy to control MOG encephalomyelitis. Neurology. 2017; 89(2):207-209.
[NPL 8] Wynford-Thomas R, Jacob A, et al. Neurological update: MOG antibody disease. J Neurol. 2019; 266(5):1280-1286.
[NPL 9] Chen J J, Flanagan E P, Bhatti M T, et al. Steroid-sparing maintenance immunotherapy for MOG-IgG associated disorder. Neurology. 2020; 95(2):el11-e120.
[NPL 10] Stiebel-Kalish H, Hellmann M A, Mimouni M, et al. Does time equal vision in the acute treatment of a cohort of AQP4 and MOG optic neuritis? Neurol Neuroimmunol Neuroinflamm. 2019; 6(4):e572.
[NPL 11] Chen J J and Bhatti M T. Clinical phenotype, radiological features, and treatment of myelin oligodendrocyte glycoprotein-immunoglobulin G (MOG-IgG) optic neuritis. Curr Opin Neurol. 2020; 33(1):47-54.
[NPL 12] Hegen H, Reindl M. Recent developments in MOG-IgG associated neurological disorders. Ther Adv Neurol Disord. 2020; 13:1756286420945135.
[NPL 13] Whittam D H, Karthikeayan V, Gibbons E, et al. Treatment of MOG antibody associated disorders: results of an international survey. J Neurol. 2020a; 267(12):3565-3577.
[NPL 14] Whittam D H, Cobo-Calvo A, Lopez-Chiriboga A S, et al. Treatment of MOG-IgG-associated disorder with rituximab: An international study of 121 patients. Mult Scler Relat Disord. 2020b; 44:102251.
[NPL 15] Durozard P, Rico A, Boutiere C, et al. Comparison of the Response to Rituximab between Myelin Oligodendrocyte Glycoprotein and Aquaporin-4 Antibody Diseases. Ann Neurol. 2020; 87(2):256-266.
[NPL 16] Hofer L S, Mariotto S, Wurth S, et al. Distinct serum and cerebrospinal fluid cytokine and chemokine profiles in autoantibody-associated demyelinating diseases. Mult Scler J Exp Transl Clin. 2019; 5(2):2055217319848463.
[NPL 17] Mult Scler Relat Disord. 2021 February; 48:102696
[NPL 18] Mult Scler Relat Disord. 2020 November; 46:102483
[NPL 19] Neurology. 2019 Apr. 16; 92(16):765-767

### SUMMARY OF INVENTION

### Technical Problem

There is no approved treatment for MOGAD or for prevention of MOGAD relapses. ISTs used empirically off-label are often only partially effective, and many are associated with numerous short- and long-term adverse effects. There is a substantial unmet need for a treatment for MOGAD and also for prevention of MOGAD relapses that would subsequently improve the long-term prognosis in patients with MOGAD.

### Solution to Problem

To solve the above-mentioned problem, the present inventors designed a phase III, randomized, double-blind (DB), placebo-controlled, multicenter study to evaluate the efficacy, safety, pharmacokinetics, and pharmacodynamics of satralizumab compared with placebo as monotherapy or in addition (add-on) to baseline/background ISTs for MOGAD relapse prevention. It is expected that the phase III study herein will effectively treat MOGAD, prevent MOGAD attacks/relapses, and reduce the risk of MOGAD attacks/relapses.

The present disclosure includes but not limited to the embodiments as exemplarily described below.

[A1.1] A medicament for treating a demyelinating disease of the central nervous system (CNS) characterized by the presence of an anti-myelin oligodendrocyte glycoprotein (MOG) antibody, or for reducing risk of relapse in a relapsing demyelinating disease of the CNS characterized by the presence of an anti-MOG antibody, in a subject who is anti-MOG antibody-positive, comprising an IL-6 inhibitor as an active ingredient.

[A1.2] The medicament of A1.1, wherein the IL-6 inhibitor is an anti-IL-6 antibody or antigen-binding fragment thereof, or an anti-IL-6 receptor antibody or antigen binding fragment thereof.

[A1.3] The medicament of A1.1 or A1.2, wherein the IL-6 inhibitor is an anti-IL-6 receptor antibody or antigen binding fragment thereof.

[A1.4] The medicament of any one of A1.1-A1.3, wherein the IL-6 inhibitor is a humanized antibody.

[A1.5] The medicament of any one of A1.1-A1.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[A1.6] The medicament of A1.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[A1.7] The medicament of A1.5 or A1.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[A1.8] The medicament of any one of A1.5-A1.7, wherein the IL-6 inhibitor is satralizumab.

[A1.9] The medicament of any one of A1.1-A1.8, for delaying relapse of, reducing frequency of relapse of, or reducing severity of relapse of the demyelinating disease of the CNS characterized by the presence of an anti-MOG antibody.

[A1.10] The medicament of any one of A1.1-A1.9, wherein the demyelinating disease of the CNS characterized by the presence of an anti-MOG antibody is a disease other than anti-aquaporin-4 (AQP4) antibody-positive NMOSD and multiple sclerosis (MS).

[A1.11] The medicament of any one of A1.1-A1.10, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[A1.12] The medicament of any one of A1.1-A1.11, wherein the demyelinating disease of the CNS characterized by the presence of an anti-MOG antibody is myelin oligodendrocyte glycoprotein antibody-associated disease (MOGAD).

[A1.13] The medicament of A1.12, wherein the MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[A1.14] The medicament of any one of A1.1-A1.13, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis, brainstem syndrome compatible with demyelination, cerebellar syndrome compatible with demyelination, and brain syndrome compatible with demyelination.

[A1.15] The medicament of any one of A1.1-A1.14, wherein the subject is anti-aquaporin-4 (AQP4) antibody-negative.

[A1.16] The medicament of any one of A1.1-A1.15, wherein the subject is aged 12 years or older.

[A1.17] The medicament of any one of A1.1-A1.16, wherein the subject is receiving no ongoing chronic immunosuppressive therapy.

[A1.18] The medicament of any one of A1.1-A1.16, wherein the subject is receiving ongoing treatment with a stable dose of azathioprine (AZA), mycophenolate mofetil (MMF), oral corticosteroid (OCS), or a combination of AZA or MMF and OCS.

[A1.19] The medicament of any one of A1.5-A1.18, which is characterized in that the medicament is used such that 60 mg or 120 mg, 120 mg or 180 mg, and 180 mg or 240 mg of the anti-IL-6 receptor antibody or antigen binding fragment thereof is administered subcutaneously to the subject with body weight of less than 40 kg, between 40 and 100 kg, and over 100 kg respectively for each administration.

[A1.20] The medicament of any one of A1.5-A1.18, which is characterized in that the medicament is used such that 60 mg of the anti-IL-6 receptor antibody or antigen binding fragment thereof is administered subcutaneously to the subject with body weight of less than 40 kg for each administration.

[A1.21] The medicament of any one of A1.5-A1.18, which is characterized in that the medicament is used such that 120 mg of the anti-IL-6 receptor antibody or antigen binding fragment thereof is administered subcutaneously to the subject with body weight of less than 40 kg for each administration.

[A1.22] The medicament of any one of A1.5-A1.18, which is characterized in that the medicament is used such that 120 mg of the anti-IL-6 receptor antibody or antigen binding fragment thereof is administered subcutaneously to the subject with body weight of between 40 and 100 kg for each administration.

[A1.23] The medicament of any one of A1.5-A1.18, which is characterized in that the medicament is used such that 180 mg of the anti-IL-6 receptor antibody or antigen binding fragment thereof is administered subcutaneously to the subject with body weight of between 40 and 100 kg for each administration.

[A1.24] The medicament of any one of A1.5-A1.18, which is characterized in that the medicament is used such that 180 mg of the anti-IL-6 receptor antibody or antigen binding fragment thereof is administered subcutaneously to the subject with body weight of over 100 kg for each administration.

[A1.25] The medicament of any one of A1.5-A1.18, which is characterized in that the medicament is used such that 240 mg of the anti-IL-6 receptor antibody or antigen binding fragment thereof is administered subcutaneously to the subject with body weight of over 100 kg for each administration.

[A1.26] The medicament of any one of A1.5-A1.25, which is characterized in that the medicament is used such that the anti-IL-6 receptor antibody or antigen binding fragment thereof is administered to the subject subcutaneously.

[A1.27] The medicament of any one of A1.5-A1.26, which is characterized in that the medicament is used such that the anti-IL-6 receptor antibody or antigen binding fragment thereof is administered to the subject every two weeks (Q2W) for three times, and thereafter every four weeks (Q4W).

[A1.28] The medicament of any one of A1.1-A1.27, which is characterized in that the medicament is used in combination with an immunosuppressive therapy (IST).

[A1.29] The medicament of A1.28, wherein the IST is a therapy with one or more immunosuppressive agents selected from the group consisting of azathioprine (AZA), mycophenolate mofetil (MMF), and oral corticosteroid (OCS).

[A1.30] The medicament of A1.29, wherein the immunosuppressive agent comprises prednisone or prednisolone.

[A1.31] The medicament of any one of A1.1-A1.30, which delays the time from an administration of the IL-6 inhibitor to the first occurrence of a relapse of the demyelinating disease of the CNS characterized by the presence of an anti-MOG antibody.

[A1.32] The medicament of A1.31, which reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[A1.33] The medicament of any one of A1.1-A1.32, which increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score, or SF-36v2 Health Survey (SF-36v2) score; or reduces the subject's Expanded Disability Status Scale (EDSS) score, Functional System Scores (FSSs) of the EDSS, Short-Form McGill Pain Questionnaire (SF-MPQ-2) score, or MOG-IgG titers.

[A2.1] A pharmaceutical composition for treating a demyelinating disease of the central nervous system (CNS) characterized by the presence of an anti-myelin oligodendrocyte glycoprotein (MOG) antibody or for reducing risk of relapse in a relapsing demyelinating disease of CNS characterized by the presence of an anti-MOG antibody in a subject who is anti-MOG antibody-positive, comprising an IL-6 inhibitor as an active ingredient.

[A2.2] The pharmaceutical composition of A2.1, wherein the IL-6 inhibitor is an anti-IL-6 antibody or antigen-binding fragment thereof, or an anti-IL-6 receptor antibody or antigen binding fragment thereof.

[A2.3] The pharmaceutical composition of A2.1 or A2.2, wherein the IL-6 inhibitor is an anti-IL-6 receptor antibody or antigen binding fragment thereof.

[A2.4] The pharmaceutical composition of any one of A2.1-A2.3, wherein the IL-6 inhibitor is a humanized antibody.

[A2.5] The pharmaceutical composition of any one of A2.1-A2.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[A2.6] The pharmaceutical composition of A2.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[A2.7] The pharmaceutical composition of A2.5 or A2.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[A2.8] The pharmaceutical composition of any one of A2.5-A2.7, wherein the IL-6 inhibitor is satralizumab.

[A2.9] The pharmaceutical composition of any one of A2.1-A2.8, for delaying relapse of, reducing frequency of relapse of, or reducing severity of relapse of the demyelinating disease of the CNS characterized by the presence of an anti-MOG antibody.

[A2.10] The pharmaceutical composition of any one of A2.1-A2.9, wherein the demyelinating disease of the CNS characterized by the presence of an anti-MOG antibody is a disease other than anti-aquaporin-4 (AQP4) antibody-positive NMOSD and multiple sclerosis (MS).

[A2.11] The pharmaceutical composition of any one of A2.1-A2.10, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[A2.12] The pharmaceutical composition of any one of A2.1-A2.11, wherein the demyelinating disease of the CNS characterized by the presence of an anti-MOG antibody is myelin oligodendrocyte glycoprotein antibody-associated disease (MOGAD).

[A2.13] The pharmaceutical composition of A2.12, wherein the MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis, brainstem syndrome compatible with demyelination, cerebellar syndrome compatible with demyelination, and brain syndrome compatible with demyelination.

[A2.14] The pharmaceutical composition of any one of A2.1-A2.13, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[A2.15] The pharmaceutical composition of any one of A2.1-A2.14, wherein the subject is anti-aquaporin-4 (AQP4) antibody-negative.

[A2.16] The pharmaceutical composition of any one of A2.1-A2.15, wherein the subject is aged 12 years or older.

[A2.17] The pharmaceutical composition of any one of A2.1-A2.16, wherein the subject is receiving no ongoing chronic immunosuppressive therapy.

[A2.18] The pharmaceutical composition of any one of A2.1-A2.16, wherein the subject is receiving ongoing treatment with a stable dose of azathioprine (AZA), mycophenolate mofetil (MMF), oral corticosteroid (OCS), or a combination of AZA or MMF and OCS.

[A2.19] The pharmaceutical composition of any one of A2.5-A2.18, which is characterized in that the pharmaceutical composition is used such that 60 mg or 120 mg, 120 mg or 180 mg, and 180 mg or 240 mg of the anti-IL-6 receptor antibody or antigen binding fragment thereof is administered subcutaneously to the subject with body weight of less than 40 kg, between 40 and 100 kg, and over 100 kg respectively for each administration.

[A2.20] The pharmaceutical composition of any one of A2.5-A2.18, which is characterized in that the pharmaceutical composition is used such that 60 mg of the anti-IL-6 receptor antibody or antigen binding fragment thereof is administered subcutaneously to the subject with body weight of less than 40 kg for each administration.

[A2.21] The pharmaceutical composition of any one of A2.5-A2.18, which is characterized in that the pharmaceutical composition is used such that 120 mg of the anti-IL-6 receptor antibody or antigen binding fragment thereof is administered subcutaneously to the subject with body weight of less than 40 kg for each administration.

[A2.22] The pharmaceutical composition of any one of A2.5-A2.18, which is characterized in that the pharmaceutical composition is used such that 120 mg of the anti-IL-6 receptor antibody or antigen binding fragment thereof is administered subcutaneously to the subject with body weight of between 40 and 100 kg for each administration.

[A2.23] The pharmaceutical composition of any one of A2.5-A2.18, which is characterized in that the pharmaceutical composition is used such that 180 mg of the anti-IL-6 receptor antibody or antigen binding fragment thereof is administered subcutaneously to the subject with body weight of between 40 and 100 kg for each administration.

[A2.24] The pharmaceutical composition of any one of A2.5-A2.18, which is characterized in that the pharmaceutical composition is used such that 180 mg of the anti-IL-6 receptor antibody or antigen binding fragment thereof is administered subcutaneously to the subject with body weight of over 100 kg for each administration.

[A2.25] The pharmaceutical composition of any one of A2.5-A2.18, which is characterized in that the pharmaceutical composition is used such that 240 mg of the anti-IL-6 receptor antibody or antigen binding fragment thereof is administered subcutaneously to the subject with body weight of over 100 kg for each administration.

[A2.26] The pharmaceutical composition of any one of A2.5-A2.25, which is characterized in that the pharmaceutical composition is used such that the anti-IL-6 receptor antibody or antigen binding fragment thereof is administered to the subject subcutaneously.

[A2.27] The pharmaceutical composition of any one of A2.5-A2.26, which is characterized in that the pharmaceutical composition is used such that the anti-IL-6 receptor antibody or antigen binding fragment thereof is administered to the subject every two weeks (Q2W) for three times, and thereafter every four weeks (Q4W).

[A2.28] The pharmaceutical composition of any one of A2.1-A2.27, which is characterized in that the pharmaceutical composition is used in combination with an immunosuppressive therapy (IST).

[A2.29] The pharmaceutical composition of A2.28, wherein the IST is a therapy with one or more immunosuppressive agents selected from the group consisting of azathioprine (AZA), mycophenolate mofetil (MMF) and oral corticosteroid (OCS).

[A2.30] The pharmaceutical composition of A2.29, wherein the immunosuppressive agent comprises prednisone or prednisolone.

[A2.31] The pharmaceutical composition of any one of A2.1-A2.30, which delays the time from an administration of the IL-6 inhibitor to the first occurrence of a relapse of the demyelinating disease of the CNS characterized by the presence of an anti-MOG antibody.

[A2.32] The pharmaceutical composition of A2.31, which reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[A2.33] The pharmaceutical composition of any one of A2.1-A2.32, which increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score, or SF-36v2 Health Survey (SF-36v2) score; or reduces the subject's Expanded Disability Status Scale (EDSS) score, Functional System Scores (FSSs) of the EDSS, Short-Form McGill Pain Questionnaire (SF-MPQ-2) score or MOG-IgG titers.

[B1] Use of an IL-6 inhibitor in the preparation of a medicament for treating demyelinating disease of the central nervous system (CNS) characterized by the presence of an anti-myelin oligodendrocyte glycoprotein (MOG) antibody or for reducing risk of relapse in a relapsing demyelinating disease of the CNS characterized by the presence of an anti-MOG antibody in a subject who is anti-MOG antibody-positive.

[B2] The use of B1, wherein the IL-6 inhibitor is an anti-IL-6 antibody or antigen-binding fragment thereof, or an anti-IL-6 receptor antibody or antigen binding fragment thereof.

[B3] The use of B1 or B2, wherein the IL-6 inhibitor is an anti-IL-6 receptor antibody or antigen binding fragment thereof.

[B4] The use of any one of B1-B3, wherein the IL-6 inhibitor is a humanized antibody.

[B5] The use of any one of B1-B4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[B6] The use of B5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[B7] The use of B5 or B6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[B8] The use of any one of B5-B7, wherein the IL-6 inhibitor is satralizumab.

[B9] The use of any one of B1-B8, wherein the medicament is for delaying relapse of, reducing frequency of relapse of, or reducing severity of relapse of the demyelinating disease of the CNS characterized by the presence of an anti-MOG antibody.

[B10] The use of any one of B1-B9, wherein the demyelinating disease of the CNS characterized by the presence of an anti-MOG antibody is a disease other than anti-aquaporin-4 (AQP4) antibody-positive NMOSD and multiple sclerosis (MS).

[B11] The use of any one of B1-B10, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[B12] The use of any one of B1-B11, wherein the demyelinating disease of the CNS characterized by the presence of an anti-MOG antibody is myelin oligodendrocyte glycoprotein antibody-associated disease (MOGAD).

[B13] The use of B12, wherein the MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[B14] The use of any one of B1-B13, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[B15] The use of any one of B1-B14, wherein the subject is anti-aquaporin-4 (AQP4) antibody-negative.

[B16] The use of any one of B1-B15, wherein the subject is aged 12 years or older.

[B17] The use of any one of B1-B16, wherein the subject is receiving no ongoing chronic immunosuppressive therapy.

[B18] The use of any one of B1-B16, wherein the subject is receiving ongoing treatment with a stable dose of azathioprine (AZA), mycophenolate mofetil (MMF), oral corticosteroid (OCS), or a combination of AZA or MMF and OCS.

[B19] The use of any one of B5-B18, wherein the medicament is characterized in that the medicament is used such that 60 mg or 120 mg, 120 mg or 180 mg, and 180 mg or 240 mg of the anti-IL-6 receptor antibody or antigen binding fragment thereof is administered subcutaneously to the subject with body weight of less than 40 kg, between 40 and 100 kg, and over 100 kg respectively for each administration.

[B20] The use of any one of B5-B18, which is characterized in that the medicament is used such that 60 mg of the anti-IL-6 receptor antibody or antigen binding fragment thereof is administered subcutaneously to the subject with body weight of less than 40 kg for each administration.

[B21] The use of any one of B5-B18, which is characterized in that the medicament is used such that 120 mg of the anti-IL-6 receptor antibody or antigen binding fragment thereof is administered subcutaneously to the subject with body weight of less than 40 kg for each administration.

[B22] The use of any one of B5-B18, which is characterized in that the medicament is used such that 120 mg of the anti-IL-6 receptor antibody or antigen binding fragment thereof is administered subcutaneously to the subject with body weight of between 40 and 100 kg for each administration.

[B23] The use of any one of B5-B18, which is characterized in that the medicament is used such that 180 mg of the anti-IL-6 receptor antibody or antigen binding fragment thereof is administered subcutaneously to the subject with body weight of between 40 and 100 kg for each administration.

[B24] The use of any one of B5-B18, which is characterized in that the medicament is used such that 180 mg of the anti-IL-6 receptor antibody or antigen binding fragment thereof is administered subcutaneously to the subject with body weight of over 100 kg for each administration.

[B25] The use of any one of B5-B18, which is characterized in that the medicament is used such that 240 mg of the anti-IL-6 receptor antibody or antigen binding fragment thereof is administered subcutaneously to the subject with body weight of over 100 kg for each administration.

[B26] The use of any one of B5-B25, wherein the medicament is characterized in that the medicament is used such that the anti-IL-6 receptor antibody or antigen binding fragment thereof is administered to the subject subcutaneously.

[B27] The use of any one of B5-B26, wherein the medicament is characterized in that the medicament is used such that the anti-IL-6 receptor antibody or antigen binding fragment thereof is administered to the subject every two weeks (Q2W) for three times, and thereafter every four weeks (Q4W).

[B28] The use of any one of B1-B27, wherein the medicament is characterized in that the medicament is used in combination with an immunosuppressive therapy (IST).

[B29] The use of B28, wherein the IST is a therapy with one or more immunosuppressive agents selected from the group consisting of azathioprine (AZA), mycophenolate mofetil (MMF), and oral corticosteroid (OCS).

[B30] The use of B29, wherein the immunosuppressive agent comprises prednisone or prednisolone.

[B31] The use of any one of B1-B30, wherein the medicament delays the time from an administration of the IL-6 inhibitor to the first occurrence of a relapse of the demyelinating disease of the CNS characterized by the presence of an anti-MOG antibody.

[B32] The use of B31, wherein the medicament reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[B33] The use of any one of B1-B32, wherein the medicament increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score, or SF-36v2 Health Survey (SF-36v2) score; or reduces the subject's Expanded Disability Status Scale (EDSS) score, Functional System Scores (FSSs) of the EDSS, Short-Form McGill Pain Questionnaire (SF-MPQ-2) score or MOG-IgG titers.

[C1] An IL-6 inhibitor for use in treating a demyelinating disease of the central nervous system (CNS) characterized by the presence of an anti-myelin oligodendrocyte glycoprotein (MOG) antibody, or for use in reducing risk of relapse in a relapsing demyelinating disease of the CNS characterized by the presence of an anti-MOG antibody in a subject who is anti-MOG antibody-positive.

[C2] The IL-6 inhibitor for use of C1, wherein the IL-6 inhibitor is an anti-IL-6 antibody or antigen-binding fragment thereof, or an anti-IL-6 receptor antibody or antigen binding fragment thereof.

[C3] The IL-6 inhibitor of C1 or C2, wherein the IL-6 inhibitor is an anti-IL-6 receptor antibody or antigen binding fragment thereof.

[C4] The IL-6 inhibitor of any one of C1-C3, wherein the IL-6 inhibitor is a humanized antibody.

[C5] The IL-6 inhibitor of any one of C1-C4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[C6] The IL-6 inhibitor for use of C5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[C7] The IL-6 inhibitor for use of C5 or C6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[C8] The IL-6 inhibitor for use of any one of C5-C7, wherein the IL-6 inhibitor is satralizumab.

[C9] The IL-6 inhibitor for use of any one of C1-C8, for delaying relapse of, reducing frequency of relapse of, or reducing severity of relapse of the demyelinating disease of the CNS characterized by the presence of an anti-MOG antibody.

[C10] The IL-6 inhibitor for use of any one of C1-C9, wherein the demyelinating disease of the CNS characterized by the presence of an anti-MOG antibody is a disease other than anti-aquaporin-4 (AQP4) antibody-positive NMOSD and multiple sclerosis (MS).

[C11] The IL-6 inhibitor for use of any one of C1-C10, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[C12] The IL-6 inhibitor for use of any one of C1-C11, wherein the demyelinating disease of the CNS characterized by the presence of an anti-MOG antibody is myelin oligodendrocyte glycoprotein antibody-associated disease (MOGAD).

[C13] The IL-6 inhibitor for use of C12, wherein the MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis, brainstem syndrome compatible with demyelination, cerebellar syndrome compatible with demyelination, and brain syndrome compatible with demyelination.

[C14] The IL-6 inhibitor for use of any one of C1-C13, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[C15] The IL-6 inhibitor for use of any one of C1-C14, wherein the subject is anti-aquaporin-4 (AQP4) antibody-negative.

[C16] The IL-6 inhibitor for use of any one of C1-C15, wherein the subject is aged 12 years or older.

[C17] The IL-6 inhibitor for use of any one of C1-C16, wherein the subject is receiving no ongoing chronic immunosuppressive therapy.

[C18] The IL-6 inhibitor for use of any one of C1-C16, wherein the subject is receiving ongoing treatment with a stable dose of azathioprine (AZA), mycophenolate mofetil (MMF), oral corticosteroid (OCS), or a combination of AZA or MMF and OCS.

[C19] The IL-6 inhibitor for use of any one of C5-C18, which is characterized in that 60 mg or 120 mg, 120 mg or 180 mg, and 180 mg or 240 mg of the anti-IL-6 receptor antibody or antigen binding fragment thereof is administered subcutaneously to the subject with body weight of less than 40 kg, between 40 and 100 kg, and over 100 kg respectively for each administration.

[C20] The IL-6 inhibitor for use of any one of C5-C18, which is characterized in that 60 mg of the anti-IL-6 receptor antibody or antigen binding fragment thereof is administered subcutaneously to the subject with body weight of less than 40 kg for each administration.

[C21] The IL-6 inhibitor for use of any one of C5-C18, which is characterized in that 120 mg of the anti-IL-6 receptor antibody or antigen binding fragment thereof is administered subcutaneously to the subject with body weight of less than 40 kg for each administration.

[C22] The IL-6 inhibitor for use of any one of C5-C18, which is characterized in that 120 mg of the anti-IL-6 receptor antibody or antigen binding fragment thereof is administered subcutaneously to the subject with body weight of between 40 and 100 kg for each administration.

[C23] The IL-6 inhibitor for use of any one of C5-C18, which is characterized in that 180 mg of the anti-IL-6 receptor antibody or antigen binding fragment thereof is administered subcutaneously to the subject with body weight of between 40 and 100 kg for each administration.

[C24] The IL-6 inhibitor for use of any one of C5-C18, which is characterized in that 180 mg of the anti-IL-6 receptor antibody or antigen binding fragment thereof is administered subcutaneously to the subject with body weight of over 100 kg for each administration.

[C25] The IL-6 inhibitor for use of any one of C5-C18, which is characterized in that 240 mg of the anti-IL-6 receptor antibody or antigen binding fragment thereof is administered subcutaneously to the subject with body weight of over 100 kg for each administration.

[C26] The IL-6 inhibitor for use of any one of C5-C25, which is characterized in that the anti-IL-6 receptor antibody or antigen binding fragment thereof is administered to the subject subcutaneously.

[C27] The IL-6 inhibitor for use of any one of C5-C26, which is characterized in that the anti-IL-6 receptor antibody or antigen binding fragment thereof is administered to the subject every two weeks (Q2W) for three times, and thereafter every four weeks (Q4W).

[C28] The IL-6 inhibitor for use of any one of C1-C27, which is used in combination with an immunosuppressive therapy (IST).

[C29] The IL-6 inhibitor for use of C28, wherein the IST is a therapy with one or more immunosuppressive agents selected from the group consisting of azathioprine (AZA), mycophenolate mofetil (MMF) and oral corticosteroid (OCS).

[C30] The IL-6 inhibitor for use of C29, wherein the immunosuppressive agent comprises prednisone or prednisolone.

[C31] The IL-6 inhibitor for use of any one of C1-C30, which delays the time from an administration of the IL-6 inhibitor to the first occurrence of a relapse of the demyelinating disease of the CNS characterized by the presence of an anti-MOG antibody.

[C32] The IL-6 inhibitor for use of C31, which reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[C33] The IL-6 inhibitor for use of any one of C1-C32, which increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score, or SF-36v2 Health Survey (SF-36v2) score; or reduces the subject's Expanded Disability Status Scale (EDSS) score, Functional System Scores (FSSs) of the EDSS, Short-Form McGill Pain Questionnaire (SF-MPQ-2) score or MOG-IgG titers.

[D1] A kit for treating a demyelinating disease of the central nervous system (CNS) characterized by the presence of an anti-myelin oligodendrocyte glycoprotein (MOG) antibody or for reducing risk of relapse in a relapsing demyelinating disease of CNS characterized by the presence of an anti-MOG antibody in a subject who is anti-MOG antibody-positive, comprising:
(1) the pharmaceutical composition of any one of A2.1-A2.33; and
(2) a package insert or label instructing administration of the pharmaceutical composition to a subject.

[D2] A subcutaneous administration device comprising a fixed dose of 60 mg of satralizumab in a pharmaceutically acceptable excipient.

[D3] A subcutaneous administration device comprising a fixed dose of 240 mg of satralizumab in a pharmaceutically acceptable excipient.

[D4] The subcutaneous administration device of D2 or D3, wherein the device is a prefilled syringe.

[D5] The subcutaneous administration device of D2 or D3, wherein the device is an autoinjector.

[E1] A method of treating a subject having demyelinating disease of central nerve system (CNS) characterized by the presence of an anti-myelin oligodendrocyte glycoprotein (MOG) antibody, the method comprising:

administering to the subject an effective amount of an IL-6 inhibitor.

[E2] The method of E1, wherein the IL-6 inhibitor is an anti-IL-6 antibody or antigen-binding fragment thereof, or an anti-IL-6 receptor antibody or antigen binding fragment thereof.

[E3] The method of E1 or E2, wherein the IL-6 inhibitor is an anti-IL-6 receptor antibody or antigen binding fragment thereof.

[E.4] The method of any one of E1-E3, wherein the IL-6 inhibitor is a humanized antibody.

[E5] The method of any one of E1-E4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[E6] The method of E5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[E7] The method of E5 or E6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[E8] The method of any one of E5-E7, wherein the IL-6 inhibitor is satralizumab.

[E9] The method of any one of E1-E8, wherein the demyelinating disease of the CNS characterized by the presence of an anti-MOG antibody is a disease other than anti-aquaporin-4 (AQP4) antibody-positive NMOSD and multiple sclerosis (MS).

[E10] The method of any one of E1-E9, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[E11] The method of any one of E1-E10, wherein the disease is myelin oligodendrocyte glycoprotein antibody-associated disease (MOGAD).

[E12] The method of E11, wherein the subject's MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis, brainstem syndrome compatible with demyelination, cerebellar syndrome compatible with demyelination, and brain syndrome compatible with demyelination.

[E13] The method of any one of E1-E12, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[E14] The method of any one of E1-E13, wherein the subject has been determined to be anti-aquaporin-4 (AQP4) antibody-negative.

[E15] The method of any one of E1-E15, wherein the subject is aged 12 years or older.

[E16] The method of any one of E1-E16, wherein the subject is receiving no ongoing chronic immunosuppressive therapy.

[E17] The method of any one of E1-E16, wherein the subject is receiving ongoing treatment with a stable dose of azathioprine (AZA), mycophenolate mofetil (MMF), oral corticosteroid (OCS), or a combination of AZA or MMF and OCS.

[E18] The method of any one of E5-E17, wherein the subject is determined to have a body weight of less than 40 kg, and the amount of the anti-IL-6 receptor antibody or antigen binding fragment thereof administered to the subject in each administration is 60 mg.

[E19] The method of any one of E5-E17, wherein the subject is determined to have a body weight of less than 40 kg, and the amount of the anti-IL-6 receptor antibody or antigen binding fragment thereof administered to the subject in each administration is 120 mg.

[E20] The method of any one of E5-E17, wherein the subject is determined to have a body weight of between 40 and 100 kg, and the amount of the anti-IL-6 receptor antibody or antigen binding fragment thereof administered to the subject in each administration is 120 mg.

[E21] The method of any one of E5-E17, wherein the subject is determined to have a body weight of between 40 and 100 kg, and the amount of the anti-IL-6 receptor antibody or antigen binding fragment thereof administered to the subject in each administration is 180 mg.

[E22] The method of any one of E5-E17, wherein the subject is determined to have a body weight of over 100 kg, and the amount of the anti-IL-6 receptor antibody or antigen binding fragment thereof administered to the subject in each administration is 180 mg.

[E23] The method of any one of E5-E17, wherein the subject is determined to have a body weight of over 100 kg, and the amount of the anti-IL-6 receptor antibody or antigen binding fragment thereof administered to the subject in each administration is 240 mg.

[E24] The method of any one of E5-E23, wherein the anti-IL-6 receptor antibody or antigen binding fragment thereof is administered to the subject subcutaneously.

[E25] The method of any one of E5-E24, wherein the anti-IL-6 receptor antibody or antigen binding fragment thereof is administered to the subject once every two weeks (Q2W) for three times, and thereafter once every four weeks (Q4W).

[E26] The method of any one of E1-E25, wherein an immunosuppressive therapy (IST) is administered to the subject concomitantly with the IL-6 inhibitor.

[E27] The method of E26, wherein the IST comprises one or more immunosuppressive agents including at least one selected from the group consisting of azathioprine (AZA), mycophenolate mofetil (MMF), and oral corticosteroid (OCS).

[E28] The method of E16, wherein the immunosuppressive agent comprises prednisone or prednisolone.

[E29] The method of any one of E1-E28, wherein administering the IL-6 inhibitor to the subject delays the time from an administration of the IL-6 inhibitor to the first occurrence of a relapse of the demyelinating disease of the CNS characterized by the presence of an anti-MOG antibody.

[E30] The method of E29, wherein administering the IL-6 inhibitor to the subject reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[E31] The method of any one of E1-E30, wherein administering the IL-6 inhibitor to the subject increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score; or SF-36v2 Health Survey (SF-36v2) score, or reduces the subject's Expanded Disability Status Scale (EDSS) score, Functional System Scores (FSSs) of the EDSS, Short-Form McGill Pain Questionnaire (SF-MPQ-2) score or MOG-IgG titers.

[F1] A method of reducing risk of relapse in a relapsing demyelinating disease of CNS characterized by the presence of an anti-MOG antibody in a subject who is anti-MOG antibody-positive, the method comprising:
administering to the subject an amount of an IL-6 inhibitor effective for reducing the risk of relapse.

[F2] The method of F1, wherein the IL-6 inhibitor is an anti-IL-6 antibody or antigen-binding fragment thereof, or an anti-IL-6 receptor antibody or antigen binding fragment thereof.

[F3] The method of F1 or F2, wherein the IL-6 inhibitor is an anti-IL-6 receptor antibody or antigen binding fragment thereof.

[F4] The method of any one of F1-F3, wherein the IL-6 inhibitor is a humanized antibody.

[F5] The method of any one of F1-F4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[F6] The method of F5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[F7] The method of F5 or F6, wherein the IL-6 inhibitor is an antibody comprising a heavy chain comprising the amino acid sequence of SEQ ID NO: 3 and a light chain comprising the amino acid sequence of SEQ ID NO: 4.

[F8] The method of any one of F5-F7, wherein the IL-6 inhibitor is satralizumab.

[F9] The method of any one of F1-F8, wherein the demyelinating disease of the CNS characterized by the presence of an anti-MOG antibody is a disease other than anti-aquaporin-4 (AQP4) antibody-positive NMOSD and multiple sclerosis (MS).

[F10] The method of any one of F1-F9, wherein the demyelinating disease of the CNS characterized by the presence of an anti-MOG antibody is a disease other than anti-aquaporin-4 (AQP4) antibody-positive NMOSD, multiple sclerosis (MS) and anti-NMDAR autoimmune encephalitis.

[F11] The method of any one of F1-F10, wherein reducing the risk of relapse comprises delaying relapse of, reducing frequency of relapse of, reducing severity of relapse of, or reducing the need for rescue therapy for relapse of the disease in the subject.

[F12] The method of any one of F1-F11, wherein the disease is myelin oligodendrocyte glycoprotein antibody-associated disease (MOGAD).

[F13] The method of F10, wherein the subject's MOGAD is characterized by (i) serum positivity for MOG-IgG by a cell-based assay, and (ii) 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[F14] The method of any one of F1-F12, wherein (i) the subject is determined to be MOG-IgG-seropositive by a cell-based assay, and (ii) the subject has experienced 2 or more attacks of any one or more of: optic neuritis (ON); transverse myelitis (TM); or encephalitis selected from the group consisting of acute disseminated encephalomyelitis (ADEM), brainstem encephalitis, cortical encephalitis; brainstem syndrome compatible with demyelination, cerebellar syndrome compatible with demyelination, and brain syndrome compatible with demyelination.

[F15] The method of any one of F1-F14, wherein the subject has been determined to be anti-aquaporin-4 (AQP4) antibody-negative.

[F16] The method of any one of F1-F15, wherein the subject is aged 12 years or older.

[F17] The method of any one of F1-F16, wherein the subject is receiving no ongoing chronic immunosuppressive therapy.

[F18] The method of any one of F1-F17, wherein the subject is receiving ongoing treatment with a stable dose of azathioprine (AZA), mycophenolate mofetil (MMF), oral corticosteroid (OCS), or a combination of AZA or MMF and OCS.

[F19] The method of any one of F5-F18, wherein the subject is determined to have a body weight of less than 40 kg, and the amount of the anti-IL-6 receptor antibody or antigen binding fragment thereof administered to the subject in each administration is 60 mg.

[F20] The method of any one of F5-E18, wherein the subject is determined to have a body weight of less than 40 kg, and the amount of the anti-IL-6 receptor antibody or antigen binding fragment thereof administered to the subject in each administration is 120 mg.

[F21] The method of any one of F5-F18, wherein the subject is determined to have a body weight of between 40 and 100 kg, and the amount of the anti-IL-6 receptor antibody or antigen binding fragment thereof administered to the subject in each administration is 120 mg.

[F22] The method of any one of F5-F18, wherein the subject is determined to have a body weight of between 40 and 100 kg, and the amount of the anti-IL-6 receptor antibody or antigen binding fragment thereof administered to the subject in each administration is 180 mg.

[F23] The method of any one of F5-F18, wherein the subject is determined to have a body weight of over 100 kg, and the amount of the anti-IL-6 receptor antibody or antigen binding fragment thereof administered to the subject in each administration is 180 mg.

[F24] The method of any one of F5-F18, wherein the subject is determined to have a body weight of over 100 kg, and the amount of the anti-IL-6 receptor antibody or antigen binding fragment thereof administered to the subject in each administration is 240 mg.

[F25] The method of any one of F5-F24, wherein the anti-IL-6 receptor antibody or antigen binding fragment thereof is administered to the subject subcutaneously.

[F26] The method of any one of F5-F25, wherein the anti-IL-6 receptor antibody or antigen binding fragment thereof is administered to the subject once every two weeks (Q2W) for three times, and thereafter once every four weeks (Q4W).

[F27] The method of any one of F1-F26, wherein an immunosuppressive therapy (IST) is administered to the subject concomitantly with the IL-6 inhibitor.

[F28] The method of F27, wherein the IST comprises one or more immunosuppressive agents, including at least one selected from the group consisting of azathioprine (AZA), mycophenolate mofetil (MMF), and oral corticosteroid (OCS).

[F29] The method of F28, wherein the immunosuppressive agent comprises prednisone or prednisolone.

[F30] The method of any one of F1-F29, wherein administering the IL-6 inhibitor to the subject delays the time from an administration of the IL-6 inhibitor to the first occurrence of a relapse of the demyelinating disease of the CNS characterized by the presence of an anti-MOG antibody.

[F31] The method of F30, wherein administering the IL-6 inhibitor to the subject reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

[F32] The method of any one of F1-F31, wherein administering the IL-6 inhibitor to the subject increases the subject's high-contrast best corrected visual acuity (BCVA), or low-contrast visual acuity (LCVA), National Eye Institute Visual Functioning Questionnaire-25 (NEI VFQ-25) composite score or subscale scores, EuroQol EQ-5D-5L score, or SF-36v2 Health Survey (SF-36v2) score; or reduces the subject's Expanded Disability Status Scale (EDSS) score, Functional System Scores (FSSs) of the EDSS, Short-Form McGill Pain Questionnaire (SF-MPQ-2) score or MOG-IgG titers.

### Advantageous Effects of Invention

The present invention can provide a medicament (a pharmaceutical composition) comprising satralizumab for treating MOGAD, preventing MOGAD attacks/relapses, or reducing the risk of MOGAD attacks/relapses.

### BRIEF DESCRIPTION OF DRAWINGS

FIG. 1 shows the study design of this Phase III, randomized, double-blind, placebo-controlled, multicenter study.

DB=double-blind; IST=(baseline/background) immunosuppressant treatment; LA=last assessment; LO=last observation; PK=pharmacokinetic; RA=relapse assessment; RFA=relapse follow-up assessment.

Notes: Groups A and B: 1:1 randomization to satralizumab+/− IST or placebo+/− IST. Satralizumab or matching placebo will be administered based on a tiered dosing scheme based on body weight of less than 40 kg: 60 mg or 120 mg; between 40 and 100 kg: 120 mg or 180 mg; more than 100 kg: 180 mg or 240 mg.

FIG. 2 shows the predicted steady-state exposure parameters (maximum concentration (Cₘₐₓ), trough concentration (C_{trough})) and receptor occupancy (RO) values in serum following administration of 60 mg, 120 mg and 180 mg of Satralizumab every 4 weeks in patients weighing less than 40 kg (30-40 kg) patients weighing 100 kg or less (40-100 kg) and patients weighing more than 100 kg (100-160 kg), respectively.

The simulations are based on 2000 individuals. Predictions for Cₘax are shown in the top panel, C_{trough} in the middle panel (Cₜᵣ=steady-state concentration at the end of a dosing interval), and RO in the bottom panel. Points are simulated data assuming anti-drug antibody (ADA) positivity in a similar proportion of participants as was observed in neuromyelitis optica spectrum disorder (NMOSD) studies. Dashed horizontal lines have been added for reference.

The assumption in setting initial doses for this Phase III study is that the pharmacokinetics of satralizumab in MOGAD is similar to that in NMOSD.

### DESCRIPTION OF EMBODIMENTS

The present invention relates to a medicament (a pharmaceutical composition) for treating a demyelinating disease of the central nervous system (CNS) characterized by the presence of an anti-myelin oligodendrocyte glycoprotein (MOG) antibody, or for reducing risk of relapse in a relapsing demyelinating disease of the CNS characterized by the presence of an anti-MOG antibody in a subject who is anti-MOG antibody-positive, comprising an IL-6 inhibitor as an active ingredient. A relapse is defined as a new clinical episode (new or worsening, acute symptoms and clinical signs, which may be accompanied by MRI evidence of acute demyelination) appearing at least 30 days (90 days if the last attack was ADEM) after the last attack.

In another aspect, the present invention also relates to use of an IL-6 inhibitor in the preparation of a medicament for treating a demyelinating disease of the CNS characterized by the presence of an anti-MOG antibody, or reducing risk of relapse in a relapsing demyelinating disease of the CNS characterized by the presence of an anti-MOG antibody in a subject who is anti-MOG antibody-positive.

In yet another aspect, the present invention relates to an IL-6 inhibitor for use in treating a demyelinating disease of the CNS characterized by the presence of an anti-MOG antibody, or reducing risk of relapse in a relapsing demyelinating disease of the CNS characterized by the presence of an anti-MOG antibody in a subject who is anti-MOG antibody-positive.

Furthermore, the present invention also relates to a kit for treating a demyelinating disease of the CNS characterized by the presence of an anti-MOG antibody, or reducing risk of relapse in a relapsing demyelinating disease of the CNS characterized by the presence of an anti-MOG antibody, in a subject who is anti-MOG antibody-positive, which comprises a pharmaceutical composition comprising an IL-6 inhibitor, and a package insert or label instructing administration of the pharmaceutical composition to a subject.

Moreover, the present invention also relates to a method of treating a subject having a demyelinating disease of the CNS characterized by the presence of an anti-MOG antibody, or reducing risk of relapse in a relapsing demyelinating disease of the CNS characterized by the presence of an anti-MOG antibody, in a subject who is anti-MOG antibody-positive, the method comprising administering to the subject an effective amount of an IL-6 inhibitor.

An “IL-6 inhibitor” of the present disclosure is a substance that blocks signal transduction by IL-6 and inhibits the biological activities of IL-6. The IL-6 inhibitor is preferably a substance that inhibits binding between IL-6 and IL-6 receptor and/or between the IL-6/IL-6 receptor complex and gp130. Examples of an IL-6 inhibitor of the present disclosure include, but are not particularly limited to, an anti-IL-6 antibody or antigen binding fragment thereof, an anti-IL-6 receptor antibody or antigen binding fragment thereof, an anti-gp130 antibody or antigen binding fragment thereof, an IL-6 variant, a soluble IL-6 receptor variant, or a partial peptide of IL-6 or IL-6 receptor, and a low-molecular-weight substance showing a similar activity. Examples of an IL-6 inhibitor of the present disclosure may be preferably an anti-IL-6 antibody or antigen-binding fragment thereof, or an anti-IL-6 receptor antibody or antigen binding fragment thereof, more preferably an anti-IL-6 receptor antibody or antigen binding fragment thereof, optionally a humanized antibody.

In some embodiments of the present disclosure, the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10. In certain embodiments in the present disclosure, the anti-IL-6 receptor antibody or antigen binding fragment thereof comprises a VH comprising the amino acid sequence of SEQ ID NO: 1 and a VL comprising the amino acid sequence of SEQ ID NO: 2. In certain embodiments in the present disclosure, the IL-6 inhibitor is an anti-IL-6 receptor antibody comprising a heavy chain comprising the amino acid sequence of SEQ ID NO: 3 and a light chain comprising the amino acid sequence of SEQ ID NO: 4. In certain embodiments in the present disclosure, the IL-6 inhibitor is satralizumab, an anti-IL-6 receptor antibody.

In certain embodiments in the present disclosure, the demyelinating disease of the CNS characterized by the presence of an anti-MOG antibody is myelin oligodendrocyte glycoprotein antibody-associated disease (MOGAD). In certain embodiments, MOGAD is characterized by (i) serum positivity for MOG-IgG by a cell-based assay, and (ii) 2 or more attacks of any one or more of optic neuritis (ON) (e.g., chronic relapsing inflammatory optic neuropathy (CRION)), transverse myelitis (TM) (e.g., longitudinally extensive transverse myelitis (LETM), short-segment transverse myelitis (STM)), acute disseminated encephalomyelitis (ADEM), brainstem encephalitis, cortical encephalitis, brainstem syndrome compatible with demyelination, cerebellar syndrome compatible with demyelination, and brain syndrome compatible with demyelination. In certain embodiments, MOGAD is a disease other than at least one selected from the group of consisting of anti-aquaporin-4 (AQP4) antibody-positive NMOSD, multiple sclerosis (MS), and anti-NMDAR autoimmune encephalitis.

Although many of the above-described symptoms overlap with typical presentations of AQP4-positive NMOSD and MS, MOGAD is distinguished from these alternative autoimmune diseases by detection of anti-MOG-IgG in serum or CSF. Thus, in certain embodiments, the present invention can be applied to a subject who is anti-aquaporin-4 (AQP4) antibody-negative. MOG-IgG seropositivity can be determined using a cell-based assay (CBA) such as described in Lopez-Chiriboga A S et al., JAMA Neurol. 2018 Nov. 1; 75(11):1355-1363. A combination of a compatible clinical and radiologic phenotype (such as described in Jarius S et al., J Neuroinflammation. 2016; 13(1):280; and Chen J J et al., Curr Opin Neurol. 2020; 33(1):47-54) and seropositivity for MOG-IgG is required to establish the diagnosis.

Although some patients, especially children, may have a monophasic course, about 80% of adult patients exhibit a highly relapsing course (Jarius et al. J Neuroinflammation. 2016; 13(1):280; Hyun et al. J Neurol Neurosurg Psychiatry. 2017; 88(10):811-817; Mult Scler Relat Disord. 2019; 30:231-235). The proportion of adolescents with relapsing disease course is believed to be similar to adults (Bruijstens et al. Eur J Paediatr Neurol 2020b; 29:32-40; Cobo-Calvo et al. Ann Neurol. 2021; 89(1):30-41), MOGAD-associated disability is attack/relapse-driven, hence the importance of relapse prevention. The current MOGAD treatment paradigm includes the use of corticosteroids with or without intravenous immunoglobulins (IVIg) or plasma exchange (PLEX) for acute treatment of attacks, and empirically selected conventional steroid-sparing ISTs and rituximab (RTX) for relapse prevention (Stiebel-Kalish et al. Neurol Neuroimmunol Neuroinflamm. 2019; 6(4):e572; Chen et al. Neurology. 2020; 95(2):el1-e120; Chen and Bhatti Curr Opin Neurol. 2020; 33(1):47-54; Hegen and Reindl, Ther Adv Neurol Disord. 2020; 13:1756286420945135; Whittam et al. J Neurol. 2020a; 267(12):3565-3577). Most recent literature indicates that these medications, which are associated with numerous short and long-term adverse effects, are often only partially effective (Chen et al. 2020; 95(2):e111-e120, Whittam et al. Mult Scler Relat Disord. 2020b; 44:102251; Durozard et al. Ann Neurol. 2020; 87(2):256-266; Cobo-Calvo et al. Ann Neurol. 2021; 89(1):30-41).

In certain embodiments, the medicament or the pharmaceutical composition of the present invention is used in combination with an immunosuppressive therapy (IST). In certain embodiments, the IST is one or more immunosuppressive agents, for example, azathioprine (AZA), mycophenolate mofetil (MMF), and oral corticosteroid (OCS), such as prednisone and prednisolone.

In certain embodiments, the medicament or the pharmaceutical composition of the present invention can delay the time from an administration of the IL-6 inhibitor to the first occurrence of a relapse of the demyelinating disease of the CNS characterized by the presence of an anti-MOG antibody. In certain embodiments, the medicament or the pharmaceutical composition of the present invention can further reduces one or more of the followings:
(a) the rate of relapses of the demyelinating disease of the CNS characterized by the presence of an anti-MOG antibody;
(b) the rate of active lesions on MRI of the neuroaxis;
(c) the proportion of subjects receiving rescue therapy; or
(d) the rate of inpatient hospitalizations.

The demyelinating disease of the CNS characterized by the presence of an anti-MOG antibody (MOGAD) is an autoimmune disease for which achieving complete remission is difficult. Thus, even if complete remission is not achieved, alleviating or improving symptoms to a level at which minimal manifestations (MM) can be maintained, or maintaining such a state, is also included in “treating a demyelinating disease of the CNS characterized by the presence of an anti-MOG antibody”.

Severity of MOGAD can be evaluated using, e.g., MOG-IgG titers (for example in serum samples) as well as EDSS, FSS, BCVA, HCVA, LCVA, NEI VFQ-25, EQ-5D-5L, SF-36v2, and/or SF-MPQ-2. Details of these evaluations are described in Examples below. For example, for NEI VFQ-25, the composite score and subscale scores range from 0 to 100, with higher scores indicating better vision-related functioning. Thus, the pharmaceutical composition of the present invention can increase the subject's NEI VFQ-25 scores. For EQ-5D-5L, a higher score indicates better health. Thus, the pharmaceutical composition of the present invention can increase the EQ-5D-5L score. For SF-36v2, a higher score indicates better health. Thus, the pharmaceutical composition of the present invention can increase the SF-36v2 score. For SF-MPQ-2, a lower score equates to lower pain, and a higher score equates to higher pain. Thus, the pharmaceutical composition of the present invention can reduce the SF-MPQ-2 score. Higher MOG-IgG titers (for example in serum samples) may be regarded as higher risk of MOGAD attacks/relapses.

Accordingly, in certain embodiments, the present invention can reduce MOG-IgG titers and/or improve scores or points of one or more of EDSS, FSS, BCVA, LCVA, NEI VFQ-25, EQ-5D-5L, SF-36v2, and/or SF-MPQ-2 in a subject to whom the present invention has been applied compared to a subject to whom the present invention has not been applied.

Efficacy of the present invention for treatment of MOGAD or reducing risk of relapse in relapsing MOGAD can be evaluated using the above-mentioned evaluation items and by quantitatively measuring severity of MOGAD before and after applying the present invention to a subject (e.g., a patient) and confirming whether a change in severity is statistically significant. Alternatively, a change or difference in a patient group to which the present invention has been applied and in a group to which the present invention has not been applied (i.e., placebo group) may be compared. For example, one or more of the scores or points for measuring severity of MOGAD as described above can be determined in a patient as a baseline before applying the present invention; after applying the present invention for a certain period of time, MOGAD severity of the patient may be determined, and an improvement of the severity compared to the baseline may then be determined. The above-mentioned evaluation criteria may be used as standards for quantitative evaluation. In any of the above evaluation standards, when a change in scores or points in a given patient after administration compared to before applying the present invention (baseline), or a difference in scores or points between a group of patients to whom the present invention is applied and a group to whom the present invention was not applied, is statistically significant, it can be said that the present invention is effective in treating MOGAD or preventing (or decreasing the risk) of MOGAD relapses. When the degree of MOGAD of a patient is severe, some MOGAD evaluation scores or points, such as EDSS score, FSSs of the EDSS, SF-MPQ-2 score, and/or MOG-IgG titers, will be high and others will be low, and when the degree is mild, they become low. Thus, it is desirable that the change or difference in these scores or points of evaluation standards decreases. On the other hand, when the degree of MOGAD of a patient is severe, other MOGAD evaluation scores or points, such as high-contrast BCVA, LCVA, NEI VFQ-25 composite score or subscale scores, EuroQol EQ-5D-5L score, and/or SF-36v2 score, will be low, and when the degree is mild, they become high. Thus, it is desirable that the change or difference in these scores or points of evaluation standards increases.

The given period of applying the present invention (e.g., administering a medicament or a pharmaceutical composition of the present invention) for evaluating efficacy is not particularly limited and includes 1 week, 2 weeks, 4 weeks, 8 weeks, 12 weeks, 24 weeks, 48 weeks, 1 year, 2 years, 3 years, 4 years, and 5 years, and the period may be shorter or longer than the exemplified period.

In the present invention, a patient having a demyelinating disease of CNS characterized by the presence of an anti-MOG antibody may receive a treatment of the present invention (e.g., a medicament, a pharmaceutical composition, a method, or the like), e.g., every two weeks (Q2W) for three times (i.e., at time zero and again at 2 weeks and 4 weeks), and thereafter every four weeks (Q4W). In some embodiments, the patient can receive the anti-IL-6 receptor antibody or antigen binding fragment thereof contained in a medicament or a composition of the present invention via subcutaneous administration route.

In addition to a treatment of a demyelinating disease of the central nervous system (CNS) characterized by the presence of an anti-myelin oligodendrocyte glycoprotein (MOG) antibody in a subject who is anti-MOG antibody-positive of the present invention, the present invention is also used for reducing risk of relapse in a relapsing demyelinating disease of CNS characterized by the presence of an anti-MOG antibody in a subject who is anti-MOG antibody-positive, such as MOGAD. In the present invention, reduction of the risk of relapse includes but is not limited to delaying relapse of, reducing frequency of relapse of, or reducing severity of relapse of, or reducing the need for rescue therapy for relapse of the demyelinating disease of CNS characterized by the presence of an anti-MOG antibody.

An anti-IL-6 receptor antibody or antigen binding fragment thereof used in the present invention binds to an IL-6 receptor, inhibits the binding of IL-6 to an IL-6 receptor, blocks signal transduction by IL-6, and inhibits the biological activities of IL-6.

An anti-IL-6 receptor antibody used in the present invention can be obtained using known methods. In particular, an anti-IL-6 receptor antibody used in the present invention is preferably a monoclonal antibody derived from a mammal. Monoclonal antibodies derived from a mammal include those produced by a hybridoma and those produced by a host that has been transformed with an expression vector containing an antibody gene using genetic engineering methods.

Preferred examples of an “IL-6 receptor antibody” in the present invention include humanized anti-IL-6 receptor antibodies produced by modifying the variable and constant regions of tocilizumab, specifically, antibodies that comprise a heavy-chain CDR1 comprising the amino acid sequence of SEQ ID NO: 5, a heavy-chain CDR2 comprising the amino acid sequence of SEQ ID NO: 6, a heavy-chain CDR3 comprising the amino acid sequence of SEQ ID NO: 7, a light-chain CDR1 comprising the amino acid sequence of SEQ ID NO: 8, a light-chain CDR2 comprising the amino acid sequence of SEQ ID NO: 9, and a light-chain CDR3 comprising the amino acid sequence of SEQ ID NO: 10.

More preferred antibodies in the present invention include antibodies that comprise a heavy-chain variable region comprising the amino acid sequence of SEQ ID NO: 1 and a light-chain variable region comprising the amino acid sequence of SEQ ID NO: 2. Still more preferred are antibodies that comprise a heavy chain comprising the amino acid sequence of SEQ ID NO: 3 (heavy chain of satralizumab (generic name); SA237 (private name)) and a light chain comprising the amino acid sequence of SEQ ID NO: 4 (light chain of satralizumab). Satralizumab (private name: SA237) is particularly preferred.

Governmental marketing approval of satralizumab has been obtained in many countries including Japan, United States, and Europe based on the indication “prevention of relapses of neuromyelitis optica spectrum disorder (including neuromyelitis optica)”. The safety profiles identified during the international joint phase III clinical trials (SA-307JG/BN40898 study and SA-309JG/BN40900 study) targeting a population of patients with neuromyelitis optica spectrum disorder (NMOSD) and/or neuromyelitis optica (NMO) were mostly favorable. No death case was reported. The percentage of patients who experienced severe adverse events in the satralizumab group was about the same as that in the placebo group. There was no big difference between the two groups on the frequency of adverse events that led to discontinuation of administration of the test drug, or on the frequency of adverse events that led to drug withdrawal. Safety profiles were similar between the SA-309JG study, which was a single-agent test, and the SA-307JG study, which was a combined test with preexisting therapy (oral steroids and/or immunosuppressive agents).

Such antibodies can be obtained according to the methods described in WO2010/035769, WO2010/107108, WO2010/106812, and such. Specifically, antibodies can be produced using genetic recombination techniques known to those skilled in the art, based on the sequence of the above-mentioned IL-6 receptor antibody (see, for example, Borrebaeck CAK and Larrick J W, THERAPEUTIC MONOCLONAL ANTIBODIES, Published in the United Kingdom by MACMILLAN PUBLISHERS LTD, 1990). A recombinant antibody can be obtained by cloning a DNA encoding the antibody from a hybridoma or an antibody-producing cell such as an antibody-producing sensitized lymphocyte, inserting the DNA into an appropriate vector, and introducing the vector into a host (host cell) to produce the antibody.

Such antibodies can be isolated and purified using isolation and purification methods conventionally used for antibody purification, without limitation. For example, the antibodies can be isolated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectric focusing, dialysis, recrystallization, and such.

The antibodies used in the present invention may be conjugate antibodies that are bound to various molecules such as polyethylene glycol (PEG), radioactive substances, and toxins. Such conjugate antibodies can be obtained by chemically modifying the obtained antibodies. Methods for antibody modification have been already established in this field. Accordingly, the term “antibody” in the present invention encompasses such conjugate antibodies.

The antibodies used in the present invention may be antibody fragments (also referred to as an antigen binding fragment of the antibody) or modified products thereof, as long as they can be suitably used in the present invention. For example, antibody fragments include Fab, F(ab′)2, Fv, and single chain Fv (scFv) in which the Fvs of the H and L chains are linked via an appropriate linker.

Specifically, the antibody fragments are produced by treating antibodies with enzymes such as papain or pepsin, or alternatively, by constructing genes encoding these antibody fragments and introducing them into expression vectors, and then expressing the vectors in appropriate host cells (see, for example, Co, M. S. et al., J. Immunol. (1994) 152, 2968-2976; Better, M. & Horwitz, A. H., Methods in Enzymology (1989) 178, 476-496; Plueckthun, A. & Skerra, A., Methods in Enzymology (1989) 178, 497-515; Lamoyi, E., Methods in Enzymology (1989) 121, 652-663; Rousseaux, J. et al., Methods in Enzymology (1989) 121, 663-666; and Bird, R. E. et al., TIBTECH (1991) 9, 132-137).

An scFv can be obtained by linking the H-chain V region and the L-chain V region of an antibody. In this scFv, the H-chain V region and the L-chain V region are linked via a linker, preferably via a peptide linker (Huston, J. S. et al., Proc. Natl. Acad. Sci. USA (1988) 85, 5879-5883). The V regions of the H and L chains in an scFv may be derived from any of the antibodies described above. Peptide linkers for linking the V regions include, for example, an arbitrary single chain peptide consisting of 12 to 19 amino acid residues.

A DNA encoding an scFv can be obtained by amplifying a DNA portion that encodes the desired amino acid sequence in template sequences with PCR using a primer pair which defines the termini of the portion, wherein a DNA encoding an H chain or an H-chain V region and a DNA encoding an L chain or an L-chain V region of the aforementioned antibodies are used as the templates, and then further amplifying the amplified DNA portion with a DNA that encodes a peptide linker portion and a primer pair that defines both ends of the linker so that it may be linked to each of the H and L chains.

Once an scFv-encoding DNA has been prepared, an expression vector comprising the DNA and a host transformed with the expression vector can be obtained according to conventional methods. In addition, an scFv can be obtained according to conventional methods by using the host.

Similar to the above, the antibody fragments can be produced by obtaining their genes, expressing them, and then using a host.

In the present invention, “as an active ingredient” means that the ingredient is contained in the pharmaceutical composition as a primal active ingredient, and the content thereof is not limited unless specifically indicated, as long as the antibodies or antigen binding fragments thereof used for the present invention are included as medicinal ingredients.

The dose of an anti-IL-6 receptor antibody or antigen binding fragment thereof contained in a medicament or a composition of the present invention is not particularly limited, and examples include 50 to 800 mg of antibody per administration, preferably 60 to 240 mg of antibody, and more preferably 60 mg, 120 mg, 180 mg, or 240 mg of antibody per administration. The dose of an anti-IL-6 receptor antibody or antigen binding fragment thereof contained in a medicament or a composition of the present invention may vary depending on the patient's body weight. In certain embodiments of the present invention, a suitable dose of the anti-IL-6 receptor antibody or antigen binding fragment for a subject with a body weight of less than 40 kg is 60 mg or 120 mg; a suitable dose for a subject with a body weight between 40 kg and 100 kg is 120 mg or 180 mg; and a suitable dose for a subject with a body weight of over 100 kg is 180 mg or 240 mg. A medicament or a composition comprising an anti-IL-6 receptor antibody or antigen binding fragment thereof of the present invention is administered to a subject via any route, including but not limited to subcutaneously, intravenously, intramuscularly, and by infusion. A preferred embodiment is subcutaneous administration.

In certain embodiments of the present invention, two or more sequential doses of an anti-IL-6 receptor antibody or antigen binding fragment thereof contained in a medicament or a composition of the present invention is administered to a subject during an initial period, wherein the doses administered during the initial period are spaced by a first dosing interval (also referred to the dosing interval that is shorter than the routine dosing interval), for example 20 weeks, 8 weeks, 4 weeks, or two weeks; and after the final dose administration of the initial period, waiting a second dosing interval that is longer than the first dosing interval and then administering a dose of an anti-IL-6 receptor antibody or antigen binding fragment thereof contained in a medicament or a composition of the present invention to a human patient, wherein optionally multiple consecutive doses are administered after the final dose administration of the initial period, and are spaced by the second dosing interval (also referred to as a “routine dosing interval”) that is not particularly limited except that it is longer than the first dosing interval. Examples of the second dosing interval include 1 day to 24 weeks, preferably 2 weeks to 8 weeks, more preferably 3 to 5 weeks, and even more preferably 4 weeks).

In certain embodiments of the present invention, an anti-IL-6 receptor antibody or antigen binding fragment thereof contained in a medicament or a composition of the present invention is administered to a subject every two weeks (Q2W) for three times, and thereafter every four weeks (Q4W).

The preferred administration schedule for an anti-IL-6 receptor antibody or antigen binding fragment thereof contained in a medicament or a composition of the present invention can be adjusted, for example, by appropriately extending the administration interval by monitoring the conditions of the disease and changes in the blood test values.

The present invention also provides an article of manufacture such as a kit, a device, and the like for use in a method of the present invention, which contains a pharmaceutical composition or a medicament of the present invention. The pharmaceutical composition or a medicament of the present invention comprises an IL-6 inhibitor as described herein. The article of manufacture may be packaged with an additional pharmaceutically acceptable carrier or medium, or instruction manual describing how to use the kits, etc.

In one embodiment, the article of manufacture comprises a container and a label on or a package insert associated with the container. Suitable containers include, for example, bottles, vials, syringes (including a prefilled syringe and an autoinjector), IV solution bags, etc. The containers may be formed from a variety of materials such as glass or plastic. In one embodiment, the container holds a composition which is by itself or combined with another composition effective for treating, preventing and/or diagnosing the condition and may have a sterile access port (for example the container may be a syringe, autoinjector, an intravenous solution bag or a vial having a stopper pierceable by a hypodermic injection needle). At least one active ingredient in the composition is an IL-6 inhibitor, preferably an anti-IL-6 receptor antibody, and more preferably an satralizumab as described in the present disclosure.

In one embodiment, a device as the article of manufacture the present invention as described above may be a prefilled syringe for injection via any administration route such as intravenously, subcutaneously, or the like, which comprises a fixed dose of an IL-6 inhibitor, preferably an anti-IL-6 receptor antibody, and more preferably an satralizumab as described in the present disclosure in a pharmaceutically acceptable excipient. In another embodiment, the device may be an autoinjector for subcutaneous administration which comprises a fixed dose of an IL-6 inhibitor, preferably an anti-IL-6 receptor antibody, and more preferably an satralizumab as described in the present disclosure in a pharmaceutically acceptable excipient. In certain embodiments, the device such as a prefilled syringe and autoinjector may comprise 60 mg, 120 mg, 180 mg, or 240 mg of satralizumab.

In the present invention, the label or package insert indicates that the pharmaceutical composition or medicament is used for treating the condition of choice. Moreover, the article of manufacture may comprise (a) a first container with a composition contained therein, wherein the composition comprises an IL-6 inhibitor, preferably an anti-IL-6 receptor antibody, and more preferably an satralizumab as described above; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

A pharmaceutical composition or a medicament of the present invention can be formulated to produce freeze-dried formulations or solution formulations by mixing, if necessary, with suitable pharmaceutically acceptable carriers, vehicles, and such. The suitable pharmaceutically acceptable carriers and vehicles include, for example, sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. Other low-molecular-weight polypeptides, proteins such as serum albumin, gelatin, and immunoglobulins, amino acids such as glycine, glutamine, asparagine, glutamic acid, aspartic acid, methionine, arginine, and lysine, sugars and carbohydrates such as polysaccharides and monosaccharides, and sugar alcohols such as mannitol and sorbitol may also be contained in the formulation. When preparing an aqueous solution for injection, physiological saline and isotonic solutions comprising glucose and other adjuvants such as D-sorbitol, D-mannose, D-mannitol, and sodium chloride may be used; and appropriate solubilizers such as alcohol (for example, ethanol), polyalcohols (such as propylene glycol and PEG), and nonionic surfactants (such as polysorbate 80, polysorbate 20, poloxamer 188, and HCO-50) may be used in combination. By mixing hyaluronidase into the formulation, a larger fluid volume can be administered subcutaneously (Expert Opin. Drug Deliv. 2007 July; 4(4): 427-40). Furthermore, syringes may be prefilled with the pharmaceutical composition of the present invention. Solution formulations can be prepared according to the method described in WO2011/090088.

If necessary, a pharmaceutical composition or a medicament of the present invention may be encapsulated in microcapsules (e.g., those made of hydroxymethylcellulose, gelatin, and poly(methylmethacrylate)), or incorporated into colloidal drug delivery systems (e.g., liposomes, albumin microspheres, microemulsions, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also known, and such methods may be applied to the pharmaceutical compositions of the present invention (Langer et al., J. Biomed. Mater. Res. 15: 267-277 (1981); Langer, Chemtech. 12: 98-105 (1982); U.S. Pat. No. 3,773,919; European Patent Application Publication No. EP 58,481; Sidman et al., Biopolymers 22: 547-556 (1983); and EP 133,988).

An anti-IL-6 receptor antibody or antigen binding fragment thereof contained in a medicament or a composition of the present invention can be administered to a patient via any appropriate route. For example, it can be administered to a patient intravenously by bolus injection or by continuous infusion, intramuscularly, intraperitoneally, intracerebrospinally, transdermally, subcutaneously, intraarticularly, sublingually, intrasynovially, orally, by inhalation, locally, or externally, for a certain period of time. Intravenous administration or subcutaneous administration is preferred. In certain embodiments of the present invention, an anti-IL-6 receptor antibody or antigen binding fragment thereof contained in a medicament or a composition of the present invention is administered to a subject subcutaneously.

All prior art references cited herein are incorporated by reference into the present specification.

### EXAMPLES

Herein below, the present invention will be specifically described with reference to the Examples, but it is not to be construed as being limited thereto.

### Example 1: Preparation of Satralizumab (SA237)

An antibody with the generic name satralizumab (and a private name of SA237), which is an IL-6 receptor antibody described in the patent document WO 2010/035769 as comprising a heavy chain having the amino acid sequence of SEQ ID NO: 26 (SEQ ID NO: 3 in the present specification) and a light chain having the amino acid sequence of SEQ ID NO: 29 (SEQ ID NO: 4 in the present specification)), was prepared according to the description of that patent document. The amino acid sequence of the heavy chain variable region is shown in SEQ ID NO: 1, and the amino acid sequence of the light chain variable region is shown in SEQ ID NO: 2. Using the prepared antibody, a subcutaneous administration preparation was prepared by the method described in the patent document WO 2011/090088.

### Example 2: Phase III, Randomized, Double-Blind, Placebo-Controlled, Multicenter Study

### 1. Study Design

### 1.1 Overall Design

### 1.1.1 Overview of Study Design

This Phase III, randomized, double-blind (DB), placebo-controlled, multicenter study is designed to evaluate the efficacy, safety, pharmacokinetics, and pharmacodynamics of satralizumab compared with placebo as monotherapy or in addition (add-on) to baseline/background ISTs for MOGAD relapse prevention.

The study will include a screening period of up to 28 days, and an event-driven DB treatment period.

During the screening period, individuals' eligibility will be evaluated for study participation.

This study will enroll approximately 152 participants with MOGAD across all sites in a global enrollment phase.

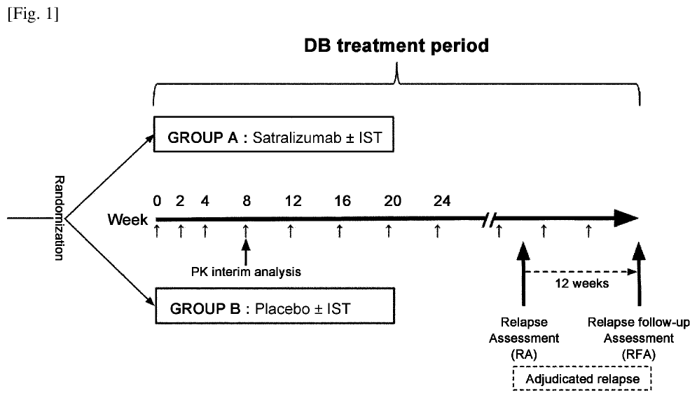

A study schema is provided in FIG. 1.

### 1.1.2 Double-Blind Treatment Period

During the DB treatment period, participants will be randomized at a 1:1 ratio to receive either satralizumab (60, 120, or 180 mg based on body weight) or placebo as monotherapy or add-on therapy to baseline/background ISTs used for MOGAD relapse prevention.

Randomization will be stratified based on the following:
Use of baseline/background ISTs
Region

Blinded study drug will be administered subcutaneously to all participants at Weeks 0, 2, 4, and every 4 weeks (Q4W) thereafter until the end of the DB treatment period.

### Pharmacokinetic Interim Analysis

An interim analysis of pharmacokinetic (PK) data will be performed during the DB treatment period.

The purpose of the interim analysis is to confirm that the achieved exposure to satralizumab (and predicted receptor occupancy [RO]) is within the target range. Based on the results from the interim PK analysis and pre-specified criteria, the study drug dose may be increased if needed to achieve target concentrations (see Section 1.3).

### Baseline/Background Immunosuppressive Therapies

The baseline/background immunosuppressive therapies permitted in this study are AZA, MMF, baseline OCS with protocol-defined OCS taper in the study, and a combination of AZA or MMF and baseline OCS with a protocol-defined OCS taper in the study.

Participants should remain on stable dose of AZA or MMF throughout the DB treatment period (except for dose reduction or discontinuation due to safety reasons, see Section 3.2.1.1).

### 1.1.3 Diagnostic Criteria for MOGAD Relapses

MOGAD relapse as defined for this study is the occurrence of new or worsening, acute neurological symptom(s) with objective changes (clinical findings or signs) on clinical (neurologic and ophthalmological) examination that persist for more than 24 hours as confirmed by the investigator. The symptoms must be attributable to MOGAD, that is, confounding clinical factors (e.g., fever, infection, injury, change in mood, adverse reactions to medications, or other neurological disorders) must be ruled out. MOGAD attacks can affect four major areas of the CNS resulting in the corresponding clinical syndromes (presentations, manifestations, or phenotypes):
The optic nerve, resulting in optic neuritis
The spinal cord, resulting in transverse myelitis
The brainstem and/or cerebellum, resulting in a brainstem/cerebellar syndrome
The brain, resulting in acute disseminated encephalomyelitis (ADEM) or other brain syndromes compatible with demyelination (tumefactive lesions, cortical disease with seizures).

These areas may be affected simultaneously during an attack (e.g., an attack can consist of simultaneous optic neuritis and transverse myelitis, or optic neuritis and ADEM).

The diagnosis of MOGAD relapses (see Table 1) in the four relevant domains/CNS regions (optic nerve, spinal cord, brainstem and/or cerebellum, brain) will be based on the criteria that involve:
Description of the new or worsening, acute neurological symptom(s) persisting for more than 24 hours
Findings on physical examination (including neurological systems) and vital signs
Functional System Scores (FSSs) of the Expanded Disability Status Scale (EDSS), as determined by an independent assessor
Ophthalmology examination results, including high-contrast visual acuity (HCVA) and low-contrast visual acuity (LCVA), assessment of the relative afferent pupillary defect (RAPD), and the appearance of the optic disc (presence of new optic disc swelling), as determined by an independent assessor
Whole neuroaxis magnetic resonance imaging (MRI) scan with gadolinium

Evidence of at least one corresponding active lesion on the MRI of the neuroaxis will be used for confirmation in cases where the clinical findings are equivocal or nonspecific.

Optic neuritis attacks are based on high-contrast best corrected visual acuity (BCVA) changes in combination with additional clinical signs, which include LCVA change, RAPD (specifically a new RAPD in the affected eye or loss of RAPD in the fellow eye), or new optic disc swelling in the affected eye. MRI to demonstrate the presence of an active lesion in the anterior visual pathway will be required if the additional clinical signs are absent or equivocal, and in situations where participant's visual acuity at the visit preceding the relapse was count-finger (CF) or worse.

Transverse myelitis attacks are based on a change in the pyramidal, sensory, or bowel and bladder FSSs of the EDSS that would be affected by this type of attack. In milder cases of myelitis, confirmation requires identification of an active lesion on the MRI of the spinal cord.

For attacks that involve the brainstem and/or the cerebellum (brainstem and/or cerebellar syndrome compatible with demyelination), changes in the brainstem and/or cerebellar FSSs in conjunction with identification of 1 or more appropriately located active MRI lesion in the brainstem and/or cerebellum will be required.

For attacks that involve the brain (brain syndromes compatible with demyelination), changes in the cerebral, sensory, or pyramidal FSSs in conjunction with identification of 1 or more appropriately located active MRI brain lesion or ADEM-specific imaging criteria (Pohl et al. 2016) will be required.

TABLE 1
Criteria for MOGAD Relapse

| Relapse Syndrome | Symptoms ^{a} (Examples) |
|---|---|
| Optic neuritis | Blurred vision, loss of vision, blind spots in vision |
| (unilateral or | Eye or retro-orbital pain/discomfort on or worsened by eye movements |
| bilateral ^{b}) | Difficulty with contrast sensitivity |
| | Difficulty perceiving colors; objects, particularly red ones, appear “washed out” |
| Transverse myelitis | Weakness in ≥1 extremity |
| | Numbness and/or paresthesias below the neck |
| | Bowel and bladder dysfunction |
| | Deep or radicular or neuropathic pain |
| | New Lhermitte's sign |
| Brainstem and | Double vision |
| cerebellar | Oscillopsia |
| syndrome | Facial numbness |
| compatible with | Facial weakness |
| demyelination | Hearing loss |
| | Vertigo |
| | Dysarthria |
| | Dysphagia |
| | Difficulties with coordination |
| Brain syndromes | ADEM: multiple neurological symptoms AND encephalopathy (alteration in |
| compatible with | consciousness) |
| demyelination | Other brain syndromes: multiple neurological symptoms |
| | Other brain syndromes: new-onset seizure |

^{a} Acute onset of new symptoms or worsening of existing symptoms that must persist for 24 hours or more.
^{b} Diagnosis of bilateral optic neuritis (ON) requires presence of symptoms, core clinical signs and additional diagnostic requirements in both eyes.

New or worsening, acute neurological symptoms and clinical signs attributable to MOGAD that occur within 30 days (or within 90 days in cases of ADEM) from the onset of a MOGAD relapse are considered the same relapse. Reoccurrence of symptoms after start of rescue therapy and not meeting criteria for a new relapse represents a so-called MOGAD flare-up episode (Bruijstens et al. 2020b; Bruijstens et al. 2020c).

The diagnosis of subsequent relapses and MOGAD flare-up episodes for an individual participant will be based on the same criteria as the first MOGAD relapse and involve the same assessments.

Relapse assessments should be performed prior to initiating any rescue therapy. For details, see Section 4.1.1.

### 1.2 Rationale for Study Design

### 1.2.1 Rationale for Study Population

The study will investigate the efficacy and safety of satralizumab in participants with MOGAD. MOG-IgG seropositivity at screening must be determined using a cell-based assay (CBA), as it is the only type of assay that allows detection of the disease-relevant anti-MOG antibodies. Alternative diagnoses with overlapping clinical features, including MS, must be excluded.

The study plans to enroll participants with an EDSS score of 0 to 6.5 and BCVA better than 20/800 in both eyes at screening, who had at least one documented MOGAD relapse within the 12 months prior to screening, or at least two attacks within the last 24 months. The participants who enter the study on a stable dose of AZA or MMF must have had a MOGAD attack while receiving the background therapy. The selection of participants with MOGAD who have evidence of recent disease activity is considered appropriate to allow for estimation of the treatment effect of satralizumab in a short timeframe and a small study population.

### 1.2.2 Rationale for Inclusion of Adolescent Participants

Inclusion of adolescent participants is supported by several arguments. Firstly, the underlying pathogenesis is considered identical in pediatric and adult patients with MOGAD, driven by peripherally produced anti-MOG-IgG that causes demyelination in the setting of the BBB disruption (Spadaro et al. 2018; Reindl and Waters 2019). Secondly, clinical phenotypes (types of MOGAD attacks/relapses) and disease course are similar in adolescent and adult patients with MOGAD. ADEM and other types of brain involvement are much less frequent in patients (adolescents and adults) above the age of 11 years (Hacohen et al. 2018, Baumann et al. 2018). In these patients, optic neuritis and transverse myelitis, in isolation or combined, are the most common types of attacks. The age-related phenotypic differences have been attributed to changing MOG expression during different stages of brain development and CNS maturation in childhood (Bruijstens et al. 2020a). Thirdly, the main types of treatment, including rescue treatment for attacks/relapses and chronic/maintenance treatment for relapse prevention, are the same in adult and pediatric patients with MOGAD.

The Phase III study requires participants to be reliable witnesses in terms of assessment of disability, pain, and general health. Adolescents are considered capable of cooperating with trial procedures.

Satralizumab has been studied in 9 adolescent patients with NMOSD aged 12 to <18 years at the time of informed consent (mean age 15 years), of whom 7 participants were randomized in the DB treatment period of Study BN40898 prior to the clinical cutoff date (CCOD) for the primary efficacy and safety reporting. The safety profile of satralizumab in these pediatric patients with NMOSD was generally consistent with the profile observed in the adult population. All adverse events reported in adolescent participants were of mild or moderate severity and resolved. None of the adolescent participants discontinued the study due to an adverse event. Data obtained in patients with NMOSD aged 12 to <18 years who received the adult dosing regimen (120 mg Q4W) showed that exposure to satralizumab was not significantly different from that in the adult population, when accounting for body weight.

### 1.2.3 Rationale for Choice of Permitted Baseline/Background Treatment

This Phase III study will enroll participants who are on no maintenance (chronic relapse prevention treatment) for MOGAD, on a stable dose of AZA or MMF with suboptimal relapse prevention, or on baseline OCS, as the aim of the study is to recruit a representative and generalizable population across treatment histories.

### 1.2.4 Rationale for Choice of Control Therapy

In this study, placebo will be used as comparator to provide objective evidence of safety and efficacy data from participants exposed to the experimental therapy. There are no therapies for MOGAD with safety and efficacy profile established in randomized controlled interventional trials. In addition, retrospective case series strongly suggest that some MS disease-modifying therapies (IFN-gamma, glatiramer acetate, teriflunomide, dimethyl fumarate, cladribine, fingolimod, natalizumab, and alemtuzumab) worsen the disease (Cobo-Calvo et al. 2021). In the absence of established effective therapy for the prevention of MOGAD relapses, the use of placebo with or without baseline/background ISTs is considered acceptable, particularly when the choice of an active comparator would imply the use of a single unproven agent. Use of baseline/background therapy will be allowed to avoid withholding of treatments that are commonly used off-label for MOGAD relapse prevention.

### 1.2.5 Rationale for Primary Endpoint Selection

The primary endpoint of time from randomization to onset of the first adjudicated relapse was chosen based on several factors: the relapsing disease course, neurological disability that is exclusively attack-driven.

### 1.2.6 Rationale for Secondary Endpoint Selection

The annualized rate of adjudicated MOGAD relapses, the annualized rate of active lesions on MRI of the neuroaxis, the proportion of participants receiving rescue therapy, and the annualized rate of inpatient hospitalizations (defined as more than an overnight stay, excluding those for elective procedures) have been selected as secondary endpoints to compare the efficacy of satralizumab versus placebo.

Prevention of relapses and the associated use of rescue therapy, and prevention of inpatient hospitalizations as a measure of healthcare utilization are meaningful goals in the chronic treatment of patients with MOGAD.

The importance of relapse prevention has been underscored by the fact that disability progression in MOGAD is considered to be solely attack-related and not driven by progression independent of relapse activity (Akaishi et al. 2021). In addition, MRI of the CNS (whole neuroaxis) is the most objective available method to study the degree and evolution of MOGAD pathology.

### 1.2.7 Rationale for Relapse Follow-Up Assessment Visit

The RFA visit will be scheduled 12 weeks after the RA visit to assess the outcomes of adjudicated MOGAD relapses. The 12-week period was defined based on the literature indicating that most recovery after an attack/relapse in demyelinating diseases, including MS and idiopathic demyelinating optic neuritis, takes place within the first 3 months (Kantarci et al. 2019, Galetta et al. 2015). While full recovery is not expected to occur in all participants within the 12-week period, this interval also allows participants in the placebo group who experienced an adjudicated relapse to transition to open-label satralizumab within a relatively short period from the relapse onset. High dose corticosteroids followed by OCS taper are routinely used to treat MOGAD attacks and to prevent flare-up episodes and rebound relapses (Jarius et al. 2016; Ramanathan et al. 2018; Brujistens et al. 2020c; Whittam et al. 2020a), therefore, continuous use of prednisone/prednisolone until the RFA visit minimizes the risk of another relapse or a flare-up before initiation of open-label satralizumab in all participants and allows for consistent assessment of relapse recovery.

### 1.2.8 Rationale for Biomarker Assessments

The study will assess whether biomarkers can aid in characterizing the mechanism of action of satralizumab in MOGAD, provide evidence of satralizumab activity in MOGAD, or increase the knowledge and understanding of MOGAD disease biology. Exploratory biomarker samples will be used for research purposes to identify pathway and/or disease biomarkers, including, but not limited to, those reflective of neuroinflammation, damage to the CNS, disease activity, or patient immune phenotype.

Pharmacodynamic (PD) biomarker samples will be collected for the assessment of target engagement (e.g., IL-6 and sIL-6R) in response to satralizumab treatment.

### 1.2.9 Rationale for Pharmacokinetic Sample Collection Schedule

Samples to assess serum concentration of satralizumab will be taken prior to each study drug administration to explore the pharmacokinetics of satralizumab in the MOGAD population. This assessment will include the impact of a range of covariates (e.g., gender, race, age, and bodyweight) on the pharmacokinetics of satralizumab, and the relationships between exposure and PD, efficacy, immunogenicity, and safety endpoints to support the recommended dose of satralizumab in the MOGAD population. PK assessments will also be used to inform the PK interim analysis to confirm the appropriate doses of satralizumab in the MOGAD population.

### 1.2.10 Rationale for Interim Pharmacokinetic Analysis

Satralizumab PK and PD data collected from the Phase I study in patients with rheumatoid arthritis were used to inform the dose-selection for Phase III studies in patients with NMOSD (120 mg Q4W), and this regimen was shown to be safe and efficacious. While the PK of satralizumab in patients with MOGAD is assumed to be similar to that in patients with NMOSD, the Sponsor is mindful that population differences in the pharmacokinetics for satralizumab are possible. Therefore, the Phase III design in MOGAD plans for an interim analysis of PK data, to ensure that participants are achieving target exposures (based on those associated with near-maximal RO in NMOSD patients) while the Sponsor remains blinded, maintaining the integrity of this pivotal study. Simulation using the existing population-PK (popPK) model (based on data in healthy volunteers and patients with NMOSD) has been used to define alternative doses in the case that the doses initially proposed do not achieve target exposures.

Further detail on the PK interim analysis is provided in Sections 1.3 and 5.4.1.

### 1.2.11 Rationale for Immunogenicity Sample Collection

Anti-drug antibodies (ADAs) were detected in a large proportion of patients with NMOSD enrolled in the Phase III studies. While impact of ADA on PK was observed, treatment benefit was not affected.

Serum samples for ADAs will be taken in parallel to PK samples, with the objective of assessing the incidence and titer-time profile of ADAs in the MOGAD population, and the impact on exposure to satralizumab, as well as on safety and efficacy. ADA data will be included in the blinded review of PK data at Week 8, for the purpose of interpretation of the satralizumab concentration data, in addition to the subsequent analysis based on the full study dataset.

### 1.2.12 Rationale for Choice of Stratification Factors

The randomization will be stratified for concomitant IST at baseline and region. These factors were selected to balance the treatment group assignment and are expected to be of prognostic value for the primary endpoint. The use of concomitant IST is included to account for potential differences in efficacy and safety depending on the treatment. Region is included to account for potential regional differences.

### 1.3 Justification for Dose and Schedule

Weight-tiered dosing via SC injection will be used in this study for the investigation of efficacy and safety of satralizumab in MOGAD as shown in Table 2.

TABLE 2
Dosing Regimen for Investigation in Phase III Study
of Satralizumab for the Treatment of MOGAD

| Body Weight at Baseline | | Dose and Regimen |
|---|---|---|
| ≥20-<40 | kg | 60 mg administered at Weeks 0, 2, 4, and Q4W thereafter as an SC injection |
| ≥40-100 | kg | 120 mg administered at Weeks 0, 2, 4, and Q4W thereafter as an SC injection |
| >100 | kg | 180 mg administered at Weeks 0, 2, 4, and Q4W thereafter as an SC injection |

PK = pharmacokinetic; Q4W = every 4 weeks.

The dosing regimen is based on a combination of sources of information, including PK, PD, and safety data for satralizumab for the initial development in NMOSD. The 120-mg fixed dosing regimen investigated in the Phase III studies in NMOSD was associated with high predicted median trough RO (95% or more) at steady-state (RO_{tr,ss}) values in most participants, and was shown to be safe and efficacious in all body weight groups. Given the anticipated similar target expression in MOGAD, exposure similar to that in NMOSD is expected to be effective also in MOGAD.

The few patients with NMOSD who had predicted RO_{tr,ss} values<80% generally had baseline body weights>100 kg, and while the safety profile was similar across bodyweight groups, the exposures in the lightest participants were in excess of those required to maintain near-maximal RO_{tr,ss}. Therefore the Sponsor has performed simulations using the existing popPK model (based on data in healthy volunteers and patients with NMOSD), to estimate the dose required to achieve the same near-maximal RO_{tr,ss} values for patients with MOGAD across the expected body weight range, maximizing the potential for efficacy. The use of the existing RO model for this purpose is considered appropriate given that the target is the same in both indications (NMOSD and MOGAD), and similar target expression is expected for MOGAD.

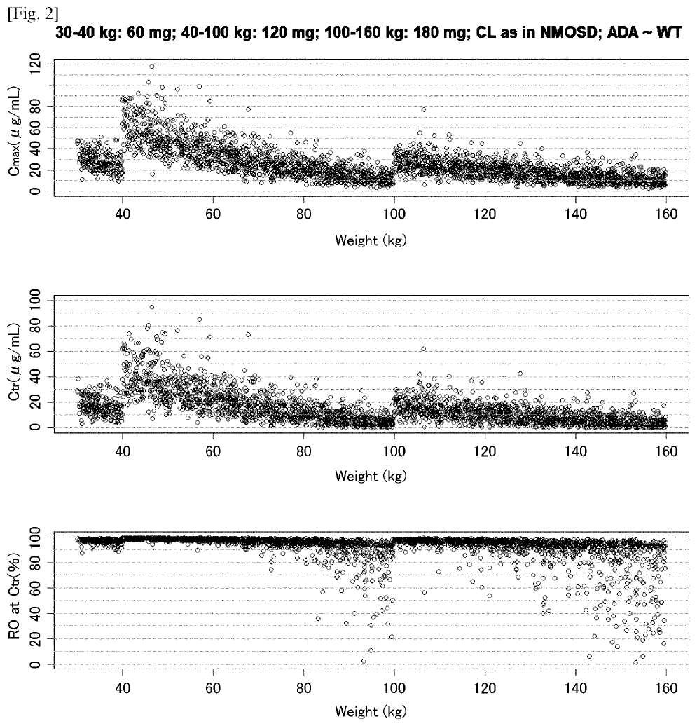

These simulations (see FIG. 2) indicate that the proposed bodyweight-tiered dose regimens would be expected to achieve similar median maximum steady state plasma drug concentration values and trough concentration at steady state values to those observed in the NMOSD Phase III studies, which were associated with near-maximal RO throughout the dose interval. The range of predicted exposures in participants weighing>100 kg receiving 180 mg does not exceed the maximum exposures achieved in the Phase III trials in NMOSD, and therefore remains within the existing exposure-safety coverage. Conversely, the predicted exposures for the 60 mg regimen in patients 20—<40 kg are expected to be sufficient to maintain high target engagement over the dose interval and avoid unnecessary overexposure.

As described, the assumption in setting initial doses for this Phase III study is that the pharmacokinetics of satralizumab in MOGAD is similar to that in NMOSD. However, the Sponsor is mindful that population differences are possible, as seen in higher total clearance (CL) values in healthy volunteers compared with the NMOSD population (covariate value [95% CI] 95.8% [67.5, 124.1]). Therefore, the proposed study design makes provision for an interim analysis of PK data (see Sections 1.2.10 and 5.4.1).

Simulation using the existing popPK model has been used to define alternative doses in the case that the doses initially proposed do not achieve target exposures. In the case that CL values in MOGAD are reflective of those in healthy volunteers (higher than those in the NMOSD population), and therefore the target exposures are not met, the dose adaptation option will be to increase the dose to the pre-defined dosing regimen of 120 mg, 180 mg, and 240 mg for participants less than 40 kg, 100 kg or less, and more than 100 kg, respectively.

In either case, the chosen dosing regimen will be associated with exposures that do not significantly exceed the existing exposure-safety coverage based on the NMOSD trials.

PK parameters in adolescent patients with NMOSD were similar to those in adult patients, and the predicted exposures resulting from the proposed bodyweight-tiered dosing regimen are supported by the existing safety profile established in the Phase III studies in adult and adolescent patients with NMOSD treated with a fixed dose of 120 mg.

### 1.4 End of Study Definition

A participant is considered to have completed the study if he or she has completed all periods of the study.

The end of this study is defined as the date of the last visit of the last participant in the study or the date at which the last data point required for SFU is received from the last participant, whichever occurs later. The end of the study is expected to occur 2.5 years after the end of the event-driven DB treatment period. In addition, the Sponsor may decide to terminate the study at any time.

### 1.5 Duration of Participation

It is estimated that participants will remain in the DB treatment period for up to 44 months.

### 2. Study Population

Approximately 152 participants with MOGAD will be enrolled in this study. The number of participants may be increased, depending on the outcome of the PK interim analysis (Section 5.4.1).

### 2.1 Inclusion Criteria

Participants are eligible to be included in the study only if the following criteria apply:
Participants who are aged 12 years or older at the time of signing Informed Consent Form
For adolescent participants: Informed Consent Form for study participation signed by the parents or a legal guardian, and assent obtained, as per local requirements
Confirmed diagnosis of MOGAD meeting the following criteria:
Documented history of 2 or more MOGAD attacks (first attack and at least 1 relapse) manifesting with the following presentations/syndromes: optic neuritis; transverse myelitis; ADEM; other brain, brainstem, or cerebellar syndrome compatible with demyelination; and any combination of the above.
Diagnosis of MOGAD attacks based on the new or worsening, acute neurologic symptoms with an objective change on neurologic and/or ophthalmologic examination (clinical signs or MRI findings in the corresponding CNS regions [i.e., optic nerve, spinal cord, brainstem, cerebellum and/or brain] or both) that persisted for more than 24 hours, AND
Serum positivity for MOG-IgG by a CBA, AND
Exclusion of alternative diagnoses, including MS.
Confirmed serum positivity for MOG-IgG at screening as assessed by a central laboratory
Body weight 20 kg or more at screening
EDSS score of 0-6.5 at screening
BCVA better than 20/800 in both eyes at screening
History of 1 or more MOGAD relapse in the 12 months prior to screening or 2 or more attacks (may include the first attack) in the 24 months prior to screening. A relapse is defined as a new clinical episode (new or worsening, acute symptoms and clinical signs, which may be accompanied by MRI evidence of acute demyelination) appearing at least 30 days (90 days if the last attack was ADEM) after the last attack.
Participants receiving either no ongoing chronic IST for MOGAD at the time of screening or receiving ongoing treatment with AZA, MMF, OCS or a combination of AZA or MMF and OCS prior to screening.
No contraindications to corticosteroids and at least one of the two other rescue treatments (IVIg or PLEX).
No contraindications to MRI (e.g., hypersensitivity to Gd-containing MRI contrast agents, implanted pacemakers, defibrillators, or other metallic objects on or inside the body that limit performing MRI scans).
For women of childbearing potential: participants who agree to remain abstinent (refrain from heterosexual intercourse) or use adequate contraception during the treatment period and for at least 3 months after the final dose of satralizumab.

### 2.2 EXCLUSION CRITERIA Exclusion Criteria Related to MOGAD

Participants are excluded from the study if any of the following criteria apply:
Presence of AQP4-IgG in the serum
History of encephalitis unrelated to MOGAD, including anti-N-methyl-d-aspartate receptor (NMDAR) encephalitis defined by the presence of anti-NMDAR antibodies in the CSF
MRI sequences typical for MS present on brain MRI at screening as assessed by the central reading center
Participants who have experienced a MOGAD relapse within 12 weeks prior to baseline, unless their EDSS is 0 at screening
Any concomitant disease other than MOGAD that may require treatment with ISTs or OCS or IV corticosteroids at doses>20 mg prednisone equivalent per day for >21 days during the study

Exclusion Criteria Related to Previous or Concomitant Therapy Participants who meet any of the following criteria related to the use of previous or concomitant therapies will be excluded from the study:
IVIg within 4 weeks prior to screening
PLEX within 4 weeks prior to screening
OCS, AZA or MMF within 4 weeks prior to screening (if not continued as baseline/background IST in the study)
Tacrolimus or cyclosporine within 6 weeks prior to screening
B-cell depleting agents, including RTX and ocrelizumab, within 6 months prior to baseline
Methotrexate within 3 months prior to screening
Neonatal Fc receptor antagonists within 6 months prior to screening
IV cyclophosphamide within 6 months prior to screening
Complement inhibitors (e.g., eculizumab) within 6 months prior to screening
Glatiramer acetate and IFN-beta within 1 month prior to screening
Fumarates (fumaric acid esters) within 2 months prior to screening
Teriflunomide within 2 years prior to screening, unless the serum/plasma concentration of teriflunomide is <0.020 micro g/mL (<20 ng/mL) prior to screening as a result of an accelerated elimination procedure for teriflunomide with cholestyramine or activated charcoal as per local prescribing information
Other MS disease-modifying treatments, including natalizumab and SIP receptor modulators (e.g., fingolimod, siponimod, ozanimod), within 6 months prior to screening
IL-6 inhibitory therapy (e.g., tocilizumab) at any time
Total body irradiation, bone marrow transplantation, and autologous hematopoietic stem cell transplantation at any time
T-cell depleting agents, including but not limited to alemtuzumab, at any time
Anti-B-lymphocyte stimulator monoclonal antibody (e.g., belimumab) at any time
Cladribine, mitoxantrone, or oral cyclophosphamide at any time
Treatment with any investigational agent within 24 weeks or 5 drug-elimination half-lives of the investigational drug prior to screening (whichever is longer)

If a retest is conducted, the last retest value before randomization must meet study criteria.

### 3. Study Treatment(s) and Concomitant Therapy

Study treatment is defined as any investigational treatment, marketed product, placebo, or medical device intended to be administered to a study participant according to the study protocol.

The IMP will be supplied by the Sponsor as prefilled syringe (PFS) for SC injection corresponding to 120 mg satralizumab. Placebo PFS is identical in composition to satralizumab PFS but does not contain the satralizumab active ingredient. It will be identical in appearance and packaging to satralizumab.

### 3.1 Study Treatments Administered

In the DB treatment period, participants will receive satralizumab or placebo at Weeks 0, 2, 4 (loading doses) and maintenance doses Q4W thereafter. The dose of study treatment will be determined on the basis of body weight. Participants will receive satralizumab according to body weight at 60 mg (less than 40 kg), 120 mg (40 to 100 kg), or 180 mg (more than 100 kg).

Study drug will be administered by SC injection in the abdominal or femoral region after all other study-related procedures have been performed at a site visit.

The dose may be modified based on the results of the interim PK analysis (see Section 1.2.10, Section 5.4.1).

### 3.2 Concomitant Therapy

### 3.2.1 Permitted Therapy

In general, investigators may manage a participant's care (including preexisting conditions) through use of supportive therapies, as clinically indicated and per local standard practice, with the exception of prohibited therapies and taking into account cautionary therapies.

### 3.2.1.1 Baseline/Background Immunosuppressive Treatment

Background treatment with any of the medications listed below is permitted.
AZA:
MMF:

### 4. Study Assessments and Procedures

### 4.1 Efficacy Assessments

### 4.1.1 Relapse Assessment

Prior to study start, participants will be trained on signs and symptoms that may be indicative of a potential relapse of optic neuritis, myelitis, or a relapse involving any other CNS region, will be instructed to remember the time of onset and duration of their symptoms related to a potential relapse, and will be asked to contact the study site immediately if they have such symptoms.

The reported episode and corresponding relapse assessment data including the following, will be submitted to the CEC regardless of the treating investigator's assessment whether the potential relapse meets the MOGAD relapse criteria defined in the protocol:
Description of the new or worsening neurological symptom(s) persisting for more than 24 hours
Findings on physical examination (including neurological systems) and vital signs
FSSs of the EDSS, as determined by the independent assessor
Ophthalmology examination results, including the HCVA and LCVA, assessment of the RAPD, and the appearance of the optic disc (presence of new optic disc swelling), as determined by an independent assessor
Whole neuroaxis MRI scan with Gd

During the DB treatment period, the independent CEC will review the data obtained at the RA visit and adjudicate if the reported episode fulfills the MOGAD relapse criteria and is a MOGAD relapse contributing to the primary endpoint. The CEC will be able to request additional information to assist in their assessment of the reported episode if deemed necessary.

### 4.1.2 Functional System Scores and Expanded Disability Status Scale Assessment

The EDSS and its associated FSSs provide a system for quantifying disability and monitoring changes in the level of disability over time. The scores in the seven Functional Systems (FS; Visual FS, Brainstem FS, Pyramidal FS, Cerebellar FS, Sensory FS, Bowel and Bladder FS, and Cerebral FS) will be used to: 1) define certain types of MOGAD relapses (see Section 1.1.3, Table 1), 2) assess severity of MOGAD relapses, and 3) assess recovery from a relapse/residual disability after a relapse. The EDSS will be used to assess recovery from a relapse/residual disability after a relapse.

The FSS/EDSS assessment will be conducted by a Neurostatus certified independent assessor (i.e., not the treating investigator). Whenever possible, the same assessor should perform the EDSS/FSS assessment for a participant throughout the study.

### 4.1.3 Ophthalmological Examination

Ophthalmological assessments will consist of HCVA and LCVA, assessment of the RAPD, and a fundus examination to assess the appearance of the optic disc.

Ophthalmological assessments will be conducted by an independent assessor (i.e., not the treating investigator), for example, an ophthalmologist, optometrist, or another site member with appropriate training and experience. Whenever possible, for each participant, all ophthalmological assessments should be performed by the same independent assessor throughout the study.

### 4.1.3.1 Assessment of Relative Afferent Pupillary Defect

For the purposes of this study, RAPD will be assessed by the swinging light test, a method of detecting differences between the two eyes in how the pupils respond to light shone in one eye at a time.

The RAPD test is used to assess unilateral or asymmetric dysfunction of the optic nerve (optic neuropathy). The physiological basis of the test is the pupillary light reflex that is consensual, i.e., a bright light shone in one eye will lead to equal constriction of both pupils. A RAPD is present if the initial consensual pupillary constriction is greater than the initial direct pupillary constriction. The RAPD results will be reported as presence or absence of RAPD in either eye.

### 4.1.3.2 Fundus Examination for Assessment of the Optic Disc Swelling

Ophthalmoscopy will be performed to assess the presence of optic disc swelling.

### 4.1.4 Magnetic Resonance Imaging

A brain MRI (without Gd) is required at screening primarily to exclude participants with imaging features that are highly specific for MS.

### 4.2 Pharmacokinetics

Serum PK samples will be collected for measurement of serum concentrations of satralizumab.

Samples may be collected at additional timepoints during the study if warranted and agreed upon between the investigator and the Sponsor.

Instructions for the collection and handling of biological samples will be provided by the Sponsor. The actual date and time (24-hour clock time) of each sample will be recorded.

Samples will be used to evaluate the pharmacokinetics of satralizumab. Samples collected for analyses of satralizumab (serum) concentration may also be used to evaluate safety or efficacy aspects related to concerns arising during or after the study.

### 4.3 Pharmacodynamics

Refer to Section 4.4 for information on PD biomarkers.

### 4.4 Biomarker Assessments

The following biomarker samples will be collected, as applicable, from participants at all sites:
Serum samples for determining eligibility at screening: autoantibodies (MOG-IgG, AQP4-IgG)
Serum samples for measuring MOG-IgG titers over time
Serum samples for measuring PD biomarkers: IL-6 and sIL-6R
Serum, plasma, and blood samples for exploratory research on biomarkers

Exploratory biomarker research may include, but will not be limited to:
Changes in the immune cells or their receptors (including but not limited to CD19+B cells and CD3+ T cells, and/or T or B cell receptors or other markers) in blood
Changes in molecular biomarkers associated with neuroinflammation in serum and/or plasma
Changes in RNA associated with neuroinflammation, disease activity, or the immune cell repertoire in blood
Changes in autoantibody titers including but not limited to MOG-IgG in serum

### 4.5 Immunogenicity Assessments

Antibodies to satralizumab will be evaluated in serum samples collected from all participants. Additionally, serum samples should be collected at the final visit from participants who discontinued study treatment or were withdrawn from the study. These samples will be tested by the Sponsor or Sponsor's designee.

Serum samples will be screened for antibodies binding to satralizumab and the titer of confirmed positive samples will be reported. Other analyses may be performed to verify the stability of antibodies to satralizumab and/or further characterize the immunogenicity of satralizumab.

The detection and characterization of antibodies to satralizumab will be performed through use of a validated assay method by or under the supervision of the Sponsor.

All samples collected for detection of antibodies to study treatment will also be evaluated for satralizumab serum concentration to enable interpretation of the antibody data.

Antibodies may be further characterized and/or evaluated for their ability to neutralize the activity of the study treatment. Samples may be stored for a maximum of 5 years (or according to local regulations) following the last participant's last visit for the study at a facility selected by the Sponsor to enable further analysis of immune responses to satralizumab.

### 4.6 Clinical Outcome Assessments

Participant-reported outcome (PRO) instruments will be completed to assess the treatment benefit of satralizumab or pharmacoeconomic evaluations as noted in the instrument description.

PRO data will be collected through use of the following instruments: NEI VFQ-25, EQ-5D-5L, SF-36v2, and SF-MPQ-2.

### 4.6.1 Data Collection Methods for Clinical Outcome Assessments

PRO instruments will be interviewer-administered at the clinic at specified timepoints during the study. At the clinic, instruments will be administered prior to any other site activities, including safety assessments and other non-PRO assessments, blood draws and the administration of study treatment, whenever possible. PRO instruments should not be administered by independent assessor(s).

PRO instruments, translated into the local language as appropriate, will be completed through use of an electronic device provided by the Sponsor. The device will be pre-programmed to enable the appropriate instruments to be administered at each specified timepoint. During clinic visits, PRO instruments should be administered as outlined below:
Participants' health status should not be discussed prior to administration of the instruments.
Sites must administer the official version of each instrument, as provided by the Sponsor. Instruments must not be copied from the protocol.
Sites should allow sufficient time for participants to complete the instruments, estimated to be 30-40 minutes at each specified visit.
The instruments should be completed in a quiet area with minimal distractions and disruptions.
Participants should be instructed to answer questions to the best of their ability; there are no right or wrong answers.
Site staff should not interpret or explain questions, but may read questions verbatim upon request.
Participants should not obtain advice or help from others (e.g., family members or friends) when completing the instruments.

### 4.6.2 Description of Clinical Outcome Assessment Instruments

### 4.6.2.1 National Eye Institute Visual Functioning Questionnaire-25 (NEI VFQ-25)

The NEI VFQ-25 captures a participant's perception of vision-related functioning and vision-related quality of life. The core measure includes 25 items that comprise 11 vision-related subscales and one item on general health (Mangione, et al. 2001). The composite score and subscale scores range from 0 to 100, with higher scores indicating better vision-related functioning. Subscale scores include General Vision, Ocular Pain, Near Activities, Distance Activities, Social Functioning, Mental Health, Role Difficulties, Dependency, Driving, Color Vision, and Peripheral Vision. The NEI VFQ-25 was validated in optic neuritis through the Optic Neuritis Treatment Trial (Cole et al. 2000). The NEI VFQ-25 takes approximately 10 minutes on average to administer in the interviewer format. The NEI VFQ-25 interviewer-administered format is available from the National Eye Institute (NEI), for example version 2000 is available online at: https://www.nei.nih.gov/sites/default/files/2019-06/vfq_ia.pdf.

### 4.6.2.2 EuroQol EQ-5D-5L

The EQ-5D-5L is a validated self-report health status questionnaire that is used to calculate a health status utility score for use in health economic analyses (EuroQol Group 1990; Brooks 1996; Herdman et al. 2011; Janssen et al. 2013). There are two components to the EQ-5D-5L: a five-item health state profile that assesses mobility, self-care, usual activities, pain/discomfort, and anxiety/depression, as well as a visual analogue scale (VAS) that measures health state. The EQ-5D-5L is designed to capture the participant's current health status. Published weighting systems allow for creation of a single composite score of the participant's health status. The EQ-5D-5L takes approximately 3 minutes to complete. It will be used in this study for informing pharmacoeconomic evaluations. The EQ-5D-5L user guide is available from the EuroQol Group, for example version 3.0 is available online at: https://euroqol.org/wp-content/uploads/2021/01/EQ-5D-5LUserguide-08-0421.pdf.

### 4.6.2.3 SF-36v2 Health Survey (SF-36v2)

The SF-36v2 is a patient-reported outcome measure assessing the participant's health-related quality of life (QoL) (Ware and Sherboume 1992). This 36-item questionnaire consists of 8 domains, Physical Functioning (10 items), Role-Physical (4 items), Bodily Pain (2 items), General Health (5 items), Vitality (4 items), Social Functioning (2 items), Role-Emotional (3 items), Mental Health (5 items), and an additional item on reported health transition. The SF-36v2 has a recall specification of 1-week and items are assessed on 3—, 5- and 6-point Likert scales. A higher score indicates better health. The SF-36v2 takes approximately 10 minutes to complete. The SF-36v2 is provided by QualityMetric Incorporated.

### 4.6.2.4 Short-Form McGill Pain Questionnaire (SF-MPQ-2)

The Short-Form McGill Pain Questionnaire (SF-MPQ-2) is a 22-item PRO measure developed in English (United States) by R. Melzack and the Initiative on Methods, Measurement, and Pain Assessment in Clinical Trials (IMMPACT) to provide a comprehensive measure of pain symptoms of both neuropathic and non-neuropathic pain conditions (Dworkin et al. 2009). The domains covered in the measure include Continuous Pain (6 items), Intermittent Pain (6 items), Neuropathic Pain (6 items), and Affective Descriptors (4 items). The recall period is “during the past week”, and each item has a response option of 0 to 10, with “none” (next to the 0) serving as one anchor and “worst possible” (next to the 10) as the other anchor. It can be scored with a global score, scored by domain score, or scored by items. A lower score equates to lower pain, and a higher score equates to higher pain. It takes approximately 10-15 minutes to complete this measure.

### 5. Statistical Considerations

### 5.1 Statistical Hypotheses

This study will compare the efficacy of satralizumab (60 mg, 120 mg, or 180 mg for participants with body weight 20 to less than 40 kg, between 40 and 100 kg, or more than 100 kg, respectively) with placebo in patients with MOGAD. Both satralizumab and placebo may be administered in addition to baseline/background therapy (see Section 1.1).

The primary endpoint is the time to first adjudicated relapse (TFR) during the DB period.

The time to adjudicated relapse and associated hazard ratio (HR) between the satralizumab and placebo group will be tested at a two-sided significance level of alpha=0.05.

The primary and secondary endpoints will be tested in a hierarchical sequence to control the overall study-wide type I error rate at the 5% significance level.

The following primary hypothesis will be tested for superiority of satralizumab over placebo at a two-sided level of alpha=0.05 with a two-sided log-rank test:

$${H}_{0} : {S}_{satralizumab} = {S}_{placebo} versus {H}_{1} : {s}_{satralizumab} \neq {S}_{placebo}$$

for which S_{satralizumab} and S_{placebo} refer to the survival function within the satralizumab- and placebo-treated participants, respectively. The Kaplan-Meier method will be used to estimate the survival functions and TFR distribution for each treatment group. A visual description of the differences between the treatment groups will be provided with Kaplan-Meier curves.

The primary analysis will be conducted based on all randomized participants if no dose adjustment is needed. In case of a dose adjustment, only participants randomized to the adjusted dose will be included in the primary analysis. For all efficacy analyses, participants will be analyzed as randomized (intention-to-treat principle). For safety analyses, all enrolled participants that received at least one dose of study treatment will be included.

### 5.2 Sample Size Determination

Approximately 152 participants will be randomly assigned to study treatment.

The purpose of this study is the estimation of the effect of satralizumab on TFR in the DB treatment period relative to placebo treatment. Point and interval estimates of the true underlying HR will be obtained.

The sample size for this study has been determined to align with the primary estimand.

The randomization will be stratified by:
Concomitant IST
Region

### 5.3 Statistical Analyses

The Statistical Analysis Plan (SAP) will be finalized prior to database lock for the primary analysis, and it will include a more technical and detailed description of the statistical analyses described in this section. This section is a summary of the planned statistical analyses of the most important endpoints, including primary and key secondary endpoints.

### 5.3.1 General Considerations

All analyses and CIs will be conducted for a two-sided significance level of 5%. Most analyses will be conducted for the DB treatment period and the All Satra treated period. The All Satra treated period is the period from the first dose of satralizumab to end of study.

For most efficacy analyses, missing data will be imputed with a reference-based multiple imputation method if not defined differently. Further details on the imputation method will be provided in the SAP.

### 5.3.2 Primary Endpoint

The primary endpoint is the time from randomization to the first occurrence of a MOGAD relapse in the DB treatment period, as determined by an adjudication committee (CEC). Point and interval estimates of the true underlying HR will be obtained. The estimand framework was used to aid the design of this study (ICH Working Group 2019).

In addition, estimates of the treatment effect will be expressed as HR and 95% CIs using a stratified Cox proportional-hazards model. If the survival function is the same between both treatment groups, the estimated HR will be 1, otherwise it will be unequal to 1.

The median TFR is not expected to be reached in this study at the time of the primary analysis; hence, every 6 months, relapse-free rates and their 95% CI will be used to describe TFR distribution in addition to the HR.

The elements of the primary estimand are defined below. The primary analysis approach and determination of sample size are aligned with this estimand.

If a participant withdraws from treatment, the reason for withdrawal will be classified as either study drug or condition related (SDCR) or not study drug or condition related (NSDCR). More details of this classification will be given in the SAP. The primary comparison will include all participants irrespective of the withdrawal reason with the assumption that participants who withdrew due to NSDCR reasons would have continued receiving their randomized treatment if they had remained in the study.
Population: participants with MOGAD as defined by the study inclusion and exclusion criteria (see Sections 2.1 and 2.2)
Primary efficacy variable: adjudicated relapse
Treatment: satralizumab vs. matching placebo

### Intercurrent Events:

Treatment with rescue therapy because of a reported episode that is not an adjudicated relapse: treatment policy
Treatment with rescue therapy before assessing the criteria for an adjudicated relapse (BCVA, FSS/EDSS, MRI, or others): treatment policy
Withdrawal from study treatment:
SDCR withdrawal from study treatment: treatment policy, data from SFU will be used if available
NSDCR withdrawal from study treatment: hypothetical, patients will be censored at withdrawal
Treatment interruption: treatment policy

Summary measure: TFR during the DB treatment period (Kaplan-Meier estimates, HR and 95% CI for treatment comparison based on the Cox proportional-hazards model and log-rank test [p-value]).

The TFR is defined as the time from randomization to the first occurrence of an adjudicated relapse during the DB period. Patients that did not experience a relapse will be censored on the day of the CCOD of the primary analysis or at withdrawal if the withdrawal was due to NSDCR reasons. For patients that withdraw due to SDCR reasons, all observed data from the DB period or SFU after withdrawal will be considered in the analysis. If these patients also withdraw from study participation, missing data will be imputed with a reference based multiple imputation method.

In addition, subgroup analyses (i.e., by region; by country for China, Japan and the United States; by baseline/background therapy group) and analyses of supplementary endpoints incorporating different approaches for the intercurrent events will be conducted. In particular, different estimand approaches such as composite estimand and hypothetical estimand will be applied to the intercurrent events that are related to rescue therapy. These analyses will be conducted as supplementary analyses to the primary analysis.

Additional details will be provided in the SAP.

### 5.3.3 Secondary Endpoints

### 5.3.3.1 Key Secondary Efficacy Endpoints

The key secondary efficacy endpoints will be tested in the following hierarchical order to control the type I error of 0.05:
Annualized rate of adjudicated MOGAD relapses
Annualized rate of active lesions on MRI of the neuroaxis
Proportion of participants receiving rescue therapy
Annualized rate of inpatient hospitalizations (defined as more than an overnight stay, excluding those for elective procedures)

The following endpoints are key secondary endpoints and described with the estimand attributes. All secondary endpoints are evaluated during the DB treatment period.

Annualized Rate of Adjudicated MOGAD Relapses during Double-Blind Treatment Period
Population: participants with MOGAD as defined by the study inclusion and exclusion criteria (see Sections 2.1 and Section 2.2)
Key secondary efficacy variable: annualized rate of adjudicated relapse (ARR) during DB treatment period
Treatment: satralizumab vs matching placebo

### Intercurrent Events:

Treatment with rescue therapy because of a reported episode that is not an adjudicated relapse: treatment policy
Treatment with rescue therapy before assessing the criteria for an adjudicated relapse (BCVA, FSS/EDSS, MRI, or others): treatment policy
Withdrawal from study treatment:
SDCR withdrawal from study treatment: treatment policy
NSDCR withdrawal from study treatment: hypothetical
Treatment interruption: treatment policy

Summary measure: Adjusted annualized relapse rates in each treatment group are compared via the rate ratio that is estimated with a negative binomial model adjusted for the stratification factors. The log-transformed time to censor or event is included as an offset variable in the analysis model. The same censoring rules as for the primary estimand will be applied. Missing data due to study withdrawal will be handled in the following way: if a participant withdraws from study due to lack of efficacy and did not have a relapse before withdrawal, a relapse will be imputed. If a participant withdraws from study due to other reasons, no data will be imputed. In both cases, the observation until the day of withdrawal will be included in the analysis.

Annualized Rate of Active Lesions on MRI of the Neuroaxis
Population: participants with MOGAD as defined by the study inclusion and exclusion criteria (see Sections 2.1 and Section 2.2)
Key secondary efficacy variable: Annualized rate of active lesions on MRI of the neuroaxis. Central reading is used to identify these lesions.
Treatment: satralizumab vs matching placebo

### Proportion of Participants Receiving Rescue Therapy During Double-Blind Treatment Period

Population: participants with MOGAD as defined by the study inclusion and exclusion criteria (see Sections 2.1 and Section 2.2)
Key secondary efficacy variable: Proportion of participants receiving rescue therapy during DB treatment period
Treatment: satralizumab vs matching placebo

### Intercurrent Events:

Treatment with rescue therapy: composite because rescue therapy use is included in the endpoint
Withdrawal from study treatment:
SDCR withdrawal from study treatment: treatment policy
NSDCR withdrawal from study treatment: hypothetical
Treatment interruption: treatment policy

Summary measure: The proportion of participants receiving rescue therapy is based on the number of participants that received rescue therapy for an episode including adjudicated MOGAD relapse. Every participant is counted once in this analysis. If a participant received rescue therapy at least once, he or she is regarded as a responder in this analysis. The proportions and associated odds ratio between the treatment groups are estimated with a logistic regression model that is adjusted for the stratification factors.

### Annualized Rate of Inpatient Hospitalizations, Defined as More Than an Overnight Stay, Excluding Those for Elective Procedures

Population: participants with MOGAD as defined by the study inclusion and exclusion criteria (see Sections 2.1 and Section 2.2)
Key secondary efficacy variable: annualized rate of inpatient hospitalizations (defined as more than an overnight stay, excluding those for elective procedures)
Treatment: satralizumab vs matching placebo

### Intercurrent Events:

Treatment of adjudicated relapses with rescue therapy within 4 days from symptom onset, which could prevent severe disability necessitating inpatient management (Stiebel-Kalish et al. 2019): treatment policy.

In addition, the timing between symptom onset and rescue medication will be investigated.
Withdrawal from study treatment:
SDCR withdrawal from study treatment: treatment policy
NSDCR withdrawal from study treatment: hypothetical
Treatment interruption: treatment policy

Summary measure: Annualized rates of inpatient hospitalizations in each treatment group are compared via the rate ratio that is estimated with a negative binomial model adjusted for the stratification factors. The log-transformed time to censor or event is included as an offset variable in the analysis model. All hospitalizations that are longer than an overnight stay and are not done for elective procedures will be counted as inpatient hospitalizations in this analysis.

### 5.3.3.2 Supplementary Secondary Endpoint

The supplementary secondary efficacy endpoint is the proportion of relapse-free participants at 6-month intervals. These proportions and corresponding 95% CIs will be based on the Kaplan-Meier curves estimated for the TFR for adjudicated relapses (primary endpoint). Intercurrent events will be handled as defined for the primary estimand.

Further details will be specified in the SAP.

### 5.3.3.3 Secondary Safety Endpoints

Safety will be assessed through summaries of exposure to study treatment, adverse events, changes from baseline in targeted clinical laboratory test results, targeted vital signs, weight, height (adolescents only), ECGs and suicidality (based on C-SSRS). All adverse event analyses will be done as proportions and rates per 100 patient-years (100PY) due to the potentially different length of the observation periods in the treatment groups.

Study treatment exposure (such as treatment duration, total dose received, and number of cycles and dose modifications, total patients years of exposure) will be summarized with descriptive statistics.

All verbatim adverse event terms will be mapped to MedDRA thesaurus terms, and adverse event severity will be graded according to NCI CTCAE v5.0. All adverse events, serious adverse events, adverse events leading to death, adverse events of special interest, and adverse events leading to study treatment discontinuation that occur on or after the first dose of study treatment (i.e., treatment-emergent adverse events) will be summarized by mapped term, appropriate thesaurus level, and severity grade. For events of varying severity, the highest grade will be used in the summaries. Deaths and cause of death will be summarized.

Relevant laboratory, vital sign (pulse rate, respiratory rate, blood pressure, pulse oximetry, and temperature), weight, and ECG data will be displayed by time, with grades identified where appropriate. Additionally, a shift table of selected laboratory tests will be used to summarize the baseline and maximum post-baseline severity grade. Changes in vital signs, weight and ECGs will be summarized.

Further details will be specified in the SAP.

### 5.3.4 Other Analyses

### 5.3.4.1 Pharmacokinetic Analyses

The PK analysis population consists of all participants in the safety analysis set with at least one valid post dose concentration result with a dosing record and sampling time. The trial will evaluate the PK characteristics of satralizumab treatment by summary statistics and non-linear mixed effects analysis (popPK).

Both the satralizumab concentration data and the results of the popPK analysis will be reported separately from the CSR.

### 5.3.4.2 Immunogenicity Analyses

The immunogenicity analysis population will consist of all participants with at least one ADA assessment. Participants will be grouped according to treatment received or, if no treatment is received prior to study discontinuation, according to treatment assigned.

The numbers and proportions of ADA-positive participants and ADA-negative participants at baseline (baseline prevalence) and after drug administration (post-baseline incidence) will be summarized by treatment group. When determining post-baseline incidence, participants are considered to be ADA-positive if they show treatment-induced ADA response or treatment-enhanced ADA response. Participants who are ADA-negative or have missing data at baseline, but develop an ADA response following study drug exposure have a treatment-induced ADA response. Participants who are ADA-positive at baseline and the titer of one or more post-baseline samples is at least 4-fold (0.60 titer unit) greater than the titer of the baseline sample have a treatment-enhanced ADA response. Participants are considered to be ADA-negative if they are ADA-negative or have missing data at baseline and all post-baseline samples are negative, or if they are ADA-positive at baseline but do not have any post-baseline samples with a titer that is at least 4-fold (0.60 titer unit) greater than the titer of the baseline sample (treatment unaffected).

The percentage of participants who have positive or negative ADA results for satralizumab will be tabulated. PK, PD, efficacy parameters, and safety will be summarized by anti-satralizumab antibody (i.e., satralizumab ADA) status.

### 5.3.4.3 Pharmacodynamic Analyses

Serum IL-6 and sIL-6R levels will be summarized by treatment group and timepoint graphically and descriptively, as appropriate.

### 5.4 Interim Analyses

### 5.4.1 Planned Interim Pharmacokinetic Analysis

An interim PK analysis will be performed during the DB treatment period. The purpose of the interim analysis is to confirm that the achieved exposures to satralizumab (and predicted RO) are within the expected target range. If the achieved exposures (and predicted RO) are not within the expected target range, the doses may be increased to 120 mg, 180 mg, and 240 mg for participants 20-<40 kg, 40-100 kg (inclusive), and >100 kg, respectively. The chosen dosing regimen will be associated with exposures that do not significantly exceed the existing exposure-safety coverage.

The iDMC will make a recommendation on whether 1) the trial can be continued with the initial dose, 2) the dose should be adapted to the pre-specified higher doses, or 3) further enrollment into the trial should be paused pending further consideration.

### 5.4.2 Optional Interim Analysis

To adapt to information that may emerge during the course of this study, the Sponsor may choose to conduct one interim efficacy analysis. This interim analysis could for example take place after results of a clinical trial for a competitor molecule become available. Below are the specifications in place to ensure the study continues to meet the highest standards of integrity when an optional interim analysis is executed.

If an interim analysis is conducted, the Sponsor will remain blinded. The interim analysis will be conducted by an external statistical group and reviewed by the iDMC. Interactions between the iDMC and Sponsor will be carried out as specified in the iDMC Charter.

The decision to conduct the optional interim analysis, along with the rationale, timing, and statistical details for the analysis, will be documented in the SAP, and the SAP will be submitted to relevant health authorities at least 2 months prior to the conduct of the interim analysis. The iDMC Charter will be updated to document potential recommendations the iDMC can make to the Sponsor as a result of the analysis (e.g., stop the study for futility), and the iDMC Charter will also be made available to relevant health authorities. The study will not be stopped for positive efficacy as a result of the interim analysis.

If there is a potential for the study to be stopped for futility as a result of the interim analysis, the threshold for declaring futility will include an assessment of the predictive probability that the specified endpoint will achieve statistical significance. Additional criteria for recommending that the study be stopped for futility may be added to the iDMC Charter. An interim analysis that might lead to stopping the study for futility will not occur before at least 50% of the information (i.e., 50% of the participants) has been accumulated.

### INDUSTRIAL APPLICABILITY

The present invention provides a means for a treatment for demyelinating disease of central nerve system (CNS) characterized by the presence of an anti-myelin oligodendrocyte glycoprotein (MOG) antibody, and also for a reduction of a risk of relapsing the demyelinating disease, comprising an anti-IL-6 receptor antibody or antigen binding fragment thereof. The present invention also provides a medicament or a pharmaceutical composition for a treatment of or a reduction of a risk of relapsing said demyelinating disease by administering an anti-IL-6 receptor antibody or antigen binding fragment thereof to a subject in need thereof. The present invention further provides a method of a treatment of, or a reduction of a risk of relapsing said demyelinating disease by administering an anti-IL-6 receptor antibody or antigen binding fragment thereof to a subject in need thereof.

### REFERENCES

Akaishi T, Misu T, Takahashi T, et al. Progression pattern of neurological disability with respect to clinical attacks in anti-MOG antibody-associated disorders. J Neuroimmunol. 2021; 351:577467.
Baumann M, Grams A, Djurdjevic T, et al. MRI of the first event in pediatric acquired demyelinating syndromes with antibodies to myelin oligodendrocyte glycoprotein. Journal of Neurology 2018; 265:845-855.
Brooks R. EuroQol: the current state of play. Health Policy 1996; 37:53-72.
Bruijstens A L, Lechner C, Flet-Berliac L, et al. E.U. paediatric MOG consortium consensus: Part 1—Classification of clinical phenotypes of paediatric myelin oligodendrocyte glycoprotein antibody-associated disorders. Eur J Paediatr Neurol. 2020a; 29:2-13.
Bruijstens A L, Breu M, Wendel E-M, et al. E.U. paediatric MOG consortium consensus: Part 4—Outcome of paediatric myelin oligodendrocyte glycoprotein antibody-associated disorders. Eur J Paediatr Neurol 2020b; 29:32-40.
Bruijstens A L, Wendel E M, Lechner C, et al. E.U. paediatric MOG consortium consensus: Part 5—Treatment of paediatric myelin oligodendrocyte glycoprotein antibody-associated disorders. Eur J Paediatr Neurol. 2020c; 29:41-53.
Burnham J A, Wright R R, Dreisbach J, Murray R S. The effect of high-dose steroids on MRI gadolinium enhancement in acute demyelinating lesions. Neurology. 1991; 41(9):1349-54.
Chen J J, Flanagan E P, Bhatti M T, et al. Steroid-sparing maintenance immunotherapy for MOG-IgG associated disorder. Neurology. 2020; 95(2):e111-e120.
Chen J J and Bhatti M T. Clinical phenotype, radiological features, and treatment of myelin oligodendrocyte glycoprotein-immunoglobulin G (MOG-IgG) optic neuritis. Curr Opin Neurol. 2020; 33(1):47-54.
Cobo-Calvo A, Ruiz A, Rollot F, et al. Clinical Features and Risk of Relapse in Children and Adults with Myelin Oligodendrocyte Glycoprotein Antibody-Associated Disease. Ann Neurol. 2021; 89(1):30-41.
Cole S R, Beck R W, Moke P S, et al. The National Eye Institute Visual Function Questionnaire: experience of the ONTT. Optic Neuritis Treatment Trial. Invest Ophthalmol Vis Sci. 2000; 41(5):1017-1021.
Durozard P, Rico A, Boutiere C, et al. Comparison of the Response to Rituximab between Myelin Oligodendrocyte Glycoprotein and Aquaporin-4 Antibody Diseases. Ann Neurol. 2020; 87(2):256-266.
Dworkin R H, Turk D C, Revicki D A, et al. Development and initial validation of an expanded and revised version of the Short-form McGill Pain Questionnaire (S F-MPQ-2). Pain. 2009; 144(1-2):35-42 EuroQol Group. EuroQol: a new facility for the measurement of health-related quality of life. Health Policy 1990; 16:199-208.
Galetta S L, Villoslada P, Levin N, et al. Acute optic neuritis: Unmet clinical needs and model for new therapies. Neurol Neuroimmunol Neuroinflamm. 2015; 2(4):e135.
Hacohen Y, Wong Y Y, Lechner C, et al. Disease Course and Treatment Responses in Children With Relapsing Myelin Oligodendrocyte Glycoprotein Antibody-Associated Disease. JAMA Neurol. 2018; 75(4):478-487.
Hegen H, Reindl M. Recent developments in MOG-IgG associated neurological disorders. Ther Adv Neurol Disord. 2020; 13:1756286420945135.
Herdman M, Gudex C, Lloyd A, et al. Development and preliminary testing of the new five-level version of EQ-5D (EQ-5D-5L). Qual Life Res 2011; 20:1727-36.
Hofer L S, Mariotto S, Wurth S, et al. Distinct serum and cerebrospinal fluid cytokine and chemokine profiles in autoantibody-associated demyelinating diseases. Mult Scler J Exp Transl Clin. 2019; 5(2):2055217319848463.
Hyun J W, Woodhall M R, Kim S H, et al. Longitudinal analysis of myelin oligodendrocyte glycoprotein antibodies in CNS inflammatory diseases. J Neurol Neurosurg Psychiatry. 2017; 88(10):811-817. ICH Working Group. E9 (R1) Addendum on estimands and sensitivity analysis in clinical trials to the guideline on statistical principles for clincial trials. 2019.
Janssen M F, Pickard A S, Golicki D, et al. Measurement properties of the EQ-5D-5L compared to the EQ-5D-3L across eight patient groups: a multi-country study. Qual Life Res 2013; 22:1717-27.
Jarius S, Ruprecht K, Kleiter I, et al. MOG-IgG in NMO and related disorders: a multicenter study of 50 patients. Part 2: Epidemiology, clinical presentation, radiological and laboratory features, treatment responses, and long-term outcome. J Neuroinflammation. 2016; 13(1):280.
Kantarci O H, Zeydan B, Atkinson E J, et al. Relapse recovery: The forgotten variable in multiple sclerosis clinical trials. Neurol Neuroimmunol Neuroinflamm. 2019; 7(2):e653.
Lopez-Chiriboga A S, Majed M, Fryer J, et al. Association of MOG-IgG Serostatus With Relapse After Acute Disseminated Encephalomyelitis and Proposed Diagnostic Criteria for MOG-IgG-Associated Disorders. JAMA Neurol. 2018 Nov. 1; 75(11):1355-1363.
Mangione C M, Lee P P, Gutierrez P R, Spritzer K, Berry S, Hays R D; National Eye Institute Visual Function Questionnaire Field Test Investigators. Development of the 25-item National Eye Institute Visual Function Questionnaire. Arch Ophthalmol. 2001; 119(7):1050-8.
Pohl D, Alper G, Van Haren K, et al. Acute disseminated encephalomyelitis: Updates on an inflammatory CNS syndrome. Neurology. 2016; 87(9 Suppl 2):S38-45.
Ramanathan S, Mohammad S, Tantsis E, et al. Clinical course, therapeutic responses and outcomes in relapsing MOG antibody-associated demyelination. J Neurol Neurosurg Psychiatry. 2018; 89(2):127-137.
Reindl M, Waters P. Myelin oligodendrocyte glycoprotein antibodies in neurological disease. Nat Rev Neurol. 2019; 15(2):89-102.
Salama S, Pardo S, Levy M. Clinical characteristics of myelin oligodendrocyte glycoprotein antibody neuromyelitis optica spectrum disorder. Mult Scler Relat Disord. 2019; 30:231-235.
Spadaro M, Winklmeier S, Beltran E, et al. Pathogenicity of human antibodies against myelin oligodendrocyte glycoprotein. Ann Neurol. 2018; 84(2):315-328.
Stiebel-Kalish H, Hellmann M A, Mimouni M, et al. Does time equal vision in the acute treatment of a cohort of AQP4 and MOG optic neuritis? Neurol Neuroimmunol Neuroinflamm. 2019; 6(4):e572.
Ware J E Jr, Sherbourne C D. The MOS 36-item short-form health survey (SF-36). I. Conceptual framework and item selection. Med Care. 1992; 30(6):473-83. PMID: 1593914.
Whittam D H, Karthikeayan V, Gibbons E, et al. Treatment of MOG antibody associated disorders: results of an international survey. J Neurol. 2020a; 267(12):3565-3577.
Whittam D H, Cobo-Calvo A, Lopez-Chiriboga A S, et al. Treatment of MOG-IgG-associated disorder with rituximab: An international study of 121 patients. Mult Scler Relat Disord. 2020b; 44:102251.
Wildemann B, Janus S, Schwarz A, et al. Failure of alemtuzumab therapy to control MOG encephalomyelitis. Neurology. 2017; 89(2):207-209.
Wynford-Thomas R, Jacob A, et al. Neurological update: MOG antibody disease. J Neurol. 2019; 266(5):1280-1286.

### SEQUENCE LISTING

1. A medicament for treating myelin oligodendrocyte glycoprotein antibody-associated disease (MOGAD), in a subject who is anti-MOG antibody-positive, comprising an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

2. The medicament of claim 1, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

3. The medicament of claim 1 or 2, wherein the anti-IL-6 receptor antibody is an antibody comprising a heavy chain comprising the amino acid sequence of SEQ ID NO: 3 and a light chain comprising the amino acid sequence of SEQ ID NO: 4.

4. The medicament of any one of claims 1-3, wherein the anti-IL-6 receptor antibody is satralizumab.

5. The medicament of any one of claims 1-4, for delaying relapse of, reducing frequency of relapse of, reducing severity of relapse of MOGAD, or reducing the risk of relapse in a patient with MOGAD.

6. The medicament of any one of claims 1-5, wherein the MOGAD is characterized by (i) serum positivity for MOG-IgG by a cell-based assay; and (ii) 2 or more attacks of any one or more of optic neuritis (ON), transverse myelitis (TM), acute disseminated encephalomyelitis (ADEM), brainstem encephalitis, cortical encephalitis, brainstem syndrome compatible with demyelination, cerebellar syndrome compatible with demyelination, and brain syndrome compatible with demyelination.

7. The medicament of any one of claims 1-6, wherein the subject is anti-aquaporin-4 (AQP4) antibody-negative.

8. The medicament of any one of claims 1-7, which is characterized in that the medicament is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively.

9. The medicament of any one of claims 1-8, which is characterized in that the medicament is used such that the anti-IL-6 receptor antibody or antigen binding fragment thereof is administered to the subject subcutaneously.

10. The medicament of any one of claims 1-9, which is characterized in that the medicament is used such that the anti-IL-6 receptor antibody or antigen binding fragment thereof is administered to the subject every two weeks (Q2W) for three times, and thereafter every four weeks (Q4W).

11. The medicament of any one of claims 1-10, which is characterized in that the medicament is used in combination with an immunosuppressive therapy (IST).

12. The medicament of claim 11, wherein the IST is a therapy with one or more of immunosuppressive agent selected from the group consisting of azathioprine (AZA), mycophenolate mofetil (MMF) and oral corticosteroid (OCS).

13. The medicament of claim 12, wherein the immunosuppressive agent comprises prednisone or prednisolone.

14. A subcutaneous administration device comprising a fixed dose of 60 mg of satralizumab in a pharmaceutically acceptable excipient.

15. A subcutaneous administration device comprising a fixed dose of 240 mg of satralizumab in a pharmaceutically acceptable excipient.


SEQUENCE LISTING (10 sequences)

SEQ ID NO: 1 - protein, 119 aa, synthetic construct
  source 1..119: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 2 - protein, 107 aa, synthetic construct
  source 1..107: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 3 - protein, 443 aa, synthetic construct
  source 1..443: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 4 - protein, 214 aa, synthetic construct
  source 1..214: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 6 aa, synthetic construct
  source 1..6: note = An artificially synthesized sequence
HDHAWS

SEQ ID NO: 6 - protein, 16 aa, synthetic construct
  source 1..16: note = An artificially synthesized sequence
FISYSGITNYNPSLQG

SEQ ID NO: 7 - protein, 10 aa, synthetic construct
  source 1..10: note = An artificially synthesized sequence
SLARTTAMDY

SEQ ID NO: 8 - protein, 11 aa, synthetic construct
  source 1..11: note = An artificially synthesized sequence
QASTDISSHLN

SEQ ID NO: 9 - protein, 7 aa, synthetic construct
  source 1..7: note = An artificially synthesized sequence
YGSHLLS

SEQ ID NO: 10 - protein, 9 aa, synthetic construct
  source 1..9: note = An artificially synthesized sequence
GQGNRLPYT